(19) United States
(12) Patent Application Publication
(10) Pub. No.: US 2022/0280579 A1
(43) Pub. Date: Sep. 8, 2022

(54) Formulations for Prevention or Reduction of C. Difficile Infections

(71) Applicant: Sporegen Limited, London (GB)

(72) Inventor: Simon CUTTING, London (GB)

(73) Assignee: Sporegen Limited, London (GB)

(21) Appl. No.: 17/636,581

(22) PCT Filed: Aug. 19, 2020
(86) PCT No.: PCT/GB2020/051984

§ 371 Date: Feb. 18, 2022

(30) Foreign Application Priority Data
Aug. 20, 2019 (GB) 1911925.4

(51) Int. Cl. A61K 35/742; C12N 1/20; A61K 38/16; A61K 31/7008; A61P 31/04; A23L 33/00; A23L 33/135; A23L 33/18

(52) U.S. Cl. CPC A61K 35/742; C12N 1/20; A61K 38/164; A61K 31/7008; A61P 31/04; A23L 33/40; A23L 33/135; A23L 33/18; A61K 2035/115

(57) ABSTRACT

Disclosed are compositions comprising one or more microorganisms selected from Bacillus sp. and the use of such compositions for preventing or reducing C. difficile infections. The compositions may be used as probiotic food formulations.

### CROSS-REFERENCE TO RELATED APPLICATIONS

This application is a 371 National Stage filing and claims the benefit under 35 U.S.C. § 120 to International Application No. PCT/GB2020/051984, filed 19 Aug. 2020, which claims priority to Great Britain Application No. GB1911925.4, filed 20 Aug. 2019, each of which is incorporated herein by reference in its entirety.

### SEQUENCE LISTING

This application herein incorporates by reference in its entirety the Sequence Listing material in the ASCII text file named “92606 PCT Sequence Listing”, created Feb. 9, 2022 and having the size of 71 kb filed with this application via EFS-Web.

### REFERENCE TO A DEPOSIT OF BIOLOGICAL MATERIAL

This application contains a reference to a deposit of biological material, which deposit is incorporated herein by reference. For complete information see last paragraph of the description.

### FIELD OF THE INVENTION

The present invention relates to formulations able to prevent or reduce infections of Clostridioides (formerly Clostridium) species in particular C. difficile. In more detail, the invention relates to foods, food ingredients, dietary supplements, dietary supplement ingredients, medical foods, foods for special medical purposes, foods for specified health use, foods for special dietary use, health foods, Complementary Medicines; Natural Health Products, Natural Health formulations, Natural Health ingredients, and pharmaceutical products, pharmaceutical preparations, pharmaceutical formulations, and pharmaceutical ingredients comprising one or more microorganisms capable of preventing or reducing Clostridioides in particular Clostridioides difficile infections in the gastrointestinal tract of humans.

### BACKGROUND OF THE INVENTION

C. difficile infection (CDI) is a nosocomial infection mainly affecting the elderly and the young. However, studies have shown an increasing rate of CDI in young people and healthy individuals without a history of antibiotic use. Clindamycin resistance has historically been one of the largest contributing factors in the development of CDI in humans and animals. CDI rates are higher in developed countries, such as the US and UK, although antibiotics are more heavily used in developing countries. Perhaps, the distribution of hyper-virulent strains and variations in diets and hygiene contribute to this difference in rates of CDI.

The human gut microbiota contains anywhere from 100-1000 bacteria species with considerable diversity between individuals. This population is dominated by strict anaerobes, and so an important question has been how they transfer between individuals. This can now be partly explained by the remarkable finding that about 60% of genera found in the human GI-tract are spore-forming bacteria representing 30% of the total intestinal microbiota. In this study, spore-forming bacteria isolated from human faeces were cultured anaerobically and then identified using analysis of the 16S rRNA. Clearly, spores (endospores) have the ability to survive outside of the host for decades and so are well-suited for transfer to other humans.

There are two types of spore forming bacteria, aerobic and anaerobic species. Since the GI-tract is considered anoxic, it has been long assumed that anaerobic bacteria predominate and are therefore likely to be more important than the aerobic counterparts. As described herein, the inventors have appreciated that aerobic spore forming bacteria are present in the GI-tract of humans and animals, and that they are acquired from the environment and therefore form a so-called ‘allochthonous’ population. The aerobic community of spore formers is mostly Bacillus species. These aerobic spore formers are mostly unnoticed using microbiome-based methods for bacterial detection, because they often exist as spores, and thus are refractory to extraction methods. Instead, they are best detected using culture-based methods.

US 2008/0057047 mentions the use of a Bacillus amyloliquefaciens named PB6 for the prophylaxis or treatment of gastrointestinal and immuno-related diseases. The supernatant of another Bacillus amyloliquefaciens was tested for prophylactic treatment for Clostridium difficile-associated disease in a mouse model by Geeraerts et al. in Journal of Gastroenterol. Hepatol. (2013). In WO 2014/020226 supernatants of Bacillus amyloliquefaciens were tested against Clostridium butyricum, Clostridium perfringens, and Clostridium histolyticum, and a Bacillus amyloliquefaciens strain named H57 was tested against diarrhea in WO 2016/011511. WO 2019/236806 mentions three strains of Bacillus amyloliquefaciens bacteria for use against C. difficile infections or Clostridium difficile-associated disease (CDAD). Lee et al. tested in British J. Nutrition (2002) the inhibition by eight carbohydrates of Lactobacillus rhamnosus GG in a human enterocyte-like Caco-2 cell assay.

### SUMMARY OF THE INVENTION

The invention provides a composition comprising one or more microorganisms selected from Bacillus amyloliquefaciens and Bacillus subtilis strains, and/or extracellular material produced by one or more of the microorganisms, and one or more food grade ingredients.

The invention also provides a composition comprising one or more microorganisms selected from Bacillus amyloliquefaciens and Bacillus subtilis strains, and/or extracellular material produced by one or more of the microorganisms, and one or more food grade ingredients, wherein the microorganism has antimicrobial activity against pathogenic strains of Clostridioides difficile and produces two or more non-ribosomal peptides.

Preferably, the one or more microorganisms are selected from the Bacillus amyloliquefaciens strains deposited as NCIMB 42971, NCIMB 42972, NCIMB 42973, NCIMB 43392 or NCIMB 43393; and the Bacillus subtilis strain deposited as NCIMB 42974.

The composition according to the invention may be formulated as a food, food ingredient, dietary supplement, dietary supplement ingredient, medical food, food for special medical purposes, food for specified health use, food for special dietary use, health food, Complementary Medicine; Natural Health Product, Natural Health formulation, Natural Health ingredient, and pharmaceutical product, pharmaceutical preparation, pharmaceutical formulation, and pharmaceutical ingredient.

The invention also provides the use of the composition of the invention for the reduction or prevention of C. difficile infection in an individual at risk of obtaining such an infection, or reduction or prevention of C. difficile colonization in asymptomatic individuals, preferably in the gastrointestinal tract of a subject, preferably a human subject.

The invention also provides a method of reducing or preventing a C. difficile infection in an individual at risk of obtaining such an infection, or reducing or preventing C. difficile colonization in an asymptomatic individual, the method comprising administering, to an individual in need of such treatment, a therapeutically effective amount of the composition of the invention.

The invention also extends to a Bacillus strain deposited as NCIMB 42971, NCIMB 42972, NCIMB 42973, NCIMB 42974, NCIMB 43392 or NCIMB 43393.

### SHORT DESCRIPTION OF THE FIGURES

FIG. 1 shows the dosing regimen for the experiment disclosed in example 2.

FIG. 2 shows the dosing regimen for the experiment disclosed in example 3.

FIG. 3 shows the dosing regimen for the experiment disclosed in example 4.

FIG. 4 shows the dosing regimen for the experiment disclosed in example 5.

FIG. 5 shows the dosing regimen for the experiment disclosed in example 6.

FIG. 6 shows internal markers (BSA and lysozyme) run on the same column. This analysis revealed that anti-CD630 activity did not correlate with protein content. Using the in vitro assay for activity the SEC fraction gave an endpoint titre of 1/5120.

FIG. 7 shows an HPLC Chromatogram showing four peaks that were separated into four fractions (Sec0, Sec1; Sec2 and Sec3) identified as Fengycin, Surfactin, Iturin and Chlorotetaine, respectively.

### DETAILED DESCRIPTION OF THE INVENTION

The present invention relates to pathogenic bacteria, and is particularly concerned with preventing or reducing bacterial infections using novel probiotic formulations, food supplements or food compositions. The invention is especially useful for preventing or reducing infections of Clostridia species, such as Clostridioides difficile.

As used herein, the term “Clostridioides difficile”, interchangeably termed “C. difficile”, describes a Gram-positive species of spore-forming bacteria which are anaerobic, motile bacteria that are human pathogens. Clostridioides difficile was until 2016 designated “Clostridium difficile”, a term that is still often used, and is also known as Peptoclostridium difficile, C. difficile, and C. diff.

The inventors have shown that some Bacillus species produce biologically active components and are surprisingly able to prevent or reduce bacterial infections caused by C. difficile. Further, it has been discovered that foods, food ingredients, dietary supplements, dietary supplement ingredients, medical foods, foods for special medical purposes, foods for specified health use, foods for special dietary use, health foods, Complementary Medicines, Natural Health Products, Natural Health formulations, Natural Health ingredients, and pharmaceutical products, pharmaceutical preparations, pharmaceutical formulations, and pharmaceutical ingredients comprising one or more of such Bacillus species or biologically active components produced by these Bacillus species may be used to prevent or reduce C. difficile infections in an individual at risk of obtaining such an infection, or prevent or reduce C. difficile colonization in asymptomatic individuals.

Hence, in a first aspect of the invention, there are provided foods, food ingredients, dietary supplements, dietary supplement ingredients, medical foods, foods for special medical purposes, foods for specified health use, foods for special dietary use, health foods, Complementary Medicines, Natural Health Products, Natural Health formulations, Natural Health ingredients, and pharmaceutical products, pharmaceutical preparations, pharmaceutical formulations, and pharmaceutical ingredients comprising a live spore and/or a live vegetative cell of Bacillus amyloliquefaciens and/or Bacillus subtilis, or extracellular material produced by the live cell, or disrupted cell homogenate, for use in preventing or ameliorating a bacterial infection caused by C. difficile. The inventors have, surprisingly, shown that these bacteria can be used prophylactically, to prevent C. difficile in non-infected individuals, and/or to prevent or delay a sub-clinical infection of C. difficile from developing into a clinical infection requiring medical therapy. Surprisingly, and preferably, the use of allochthonous bacteria (rather than anaerobic, autochthonous bacteria) can be used to effectively prevent or ameliorate bacterial infections.

In one embodiment, it is preferred that live spores and/or vegetative cells of B. amyloliquefaciens and/or Bacillus subtilis, are used to prevent or ameliorate the bacterial infection. In yet another embodiment, spores, dead cells or cell fragments or culture supernatants of B. amyloliquefaciens and/or B. subtilis, are used to prevent or reduce the bacterial infection.

However, in a most preferred embodiment, spores or live vegetative cells of B. amyloliquefaciens and/or B. subtilis, or extracellular material produced by the live cells, are used to prevent or ameliorate the infection. As described in the Examples, the inventors have shown that the supernatant extract (which does not include vegetative cells or spores, i.e., a cell-free sample) surprisingly exhibits antibacterial activity against C. difficile. Thus, in another preferred embodiment, a cell-free sample (e.g., the supernatant) comprising extracellular material produced by the live vegetative cell or disrupted cell homogenate may be used to combat the bacterial infection.

In another embodiment, the microorganism of the invention may have antimicrobial activity against pathogenic strains of Clostridioides difficile and produce one or more non-ribosomal peptides.

As used herein, the term “non-ribosomal peptides” (also known as nonribosomal peptides or NRP) means a class of peptide secondary metabolites that are synthesized by non-ribosomal peptide synthetases. Non-ribosomal peptides of the invention include, but are not limited to, peptides that are members of the Fengycin family, members of the Surfactin family, members of the Iturin family and Chlorotetaine.

The non-ribosomal peptides may be one or more peptides selected from the group consisting of a member of the Fengycin family; a member of the Surfactin family; a member of the Iturin family and Chlorotetaine. Preferably, the non-ribosomal peptides may be one or more peptides selected from the group consisting of: a member of the Fengycin family; a member of the Surfactin family and Chlorotetaine. Most preferably, the microorganism may produce the non-ribosomal peptides: a member of the Fengycin family, a member of the Surfactin family, and Chlorotetaine.

The two or more non-ribosomal peptides may be selected from the group consisting of: a member of the Fengycin family; a member of the Surfactin family; a member of the Iturin family; and Chlorotetaine. The two or more non-ribosomal peptides may be selected from the group consisting of: a member of the Fengycin family; a member of the Surfactin family; and Chlorotetaine. The microorganism may produce three or more non-ribosomal peptides. Preferably, the non-ribosomal peptides are: a member of the Fengycin family; a member of the Surfactin family; and Chlorotetaine.

In one embodiment, the general formula for members of the Fengycin family may be set out in formula I below, wherein R1 to R3 is any amino acid, and preferably R1 is L or D Tyr, R2 is Ala or Val and R3 is L or D Tyr:

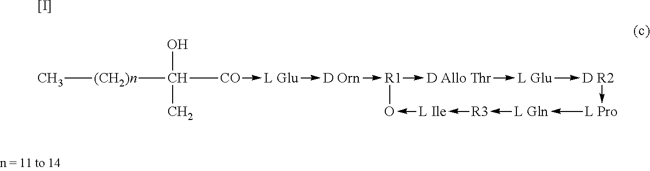

The member of the Fengycin family may be selected from a group consisting of: Fengycin A [SEQ ID NO: 11], Fengycin B [SEQ ID NO: 12], Plipastatin A [SEQ ID NO: 13] and Plipastatin B [SEQ ID NO: 14].

| | | |
|---|---|---|
| Fengycin A** | L-Glu-D-Orn-D-Tyr-D-aThr-L-Glu-D-Ala-L-Pro-L-Gln-L-Tyr-L-Ile | [symbol] - [symbol] , i- [symbol] , n- [symbol] |
| Fengycin B** | L-Glu-D-Orn-D-Tyr-D-aThr-L-Glu-D-Val-L-Pro-L-Gln-L-Tyr-L-Ile | [symbol] - [symbol] , i- [symbol] , n- [symbol] , [symbol] |
| Plipastatin A | L-Glu-D-Orn-L-Tyr-D-aThr-L-Glu-D-Ala-L-Pro-L-Gln-D-Tyr-L-Ile | [symbol] - [symbol] , [symbol] - [symbol] |
| Plipastatin B | L-Glu-D-Orn-L-Tyr-D-aThr-L-Glu-D-Val-L-Pro-L-Gln-D-Tyr-L-Ile | [symbol] - [symbol] , [symbol] - [symbol] |

[symbol]

indicates data missing or illegible when filed

Preferably, the member of the Fengycin family comprises Fengycin A, or an active derivative thereof. The Fengycin A, or active derivative thereof, may be the C₁₅, C₁₆, C₁₇ or C₁₈ isoform. Most preferably, the Fengycin A is the C₁₅ Fengycin A isoform. The Fengycin A, or active derivative thereof, may be acetylated.

In one embodiment, Fengycin A may have an amino acid sequence as set out in SEQ ID NO: 11:

[SEQ ID NO 11]
L-Glu-D-Orn-D-Tyr-D-aThr-L-Glu-D-Ala-L-Pro-L-Gln-
L-Tyr-L-Ile

Preferably, the member of the Fengycin family comprises Fengycin B, or an active derivative thereof. The Fengycin B, or active derivative thereof, may be the C₁₃, C₁₄, C₁₅ or C₁₆ isoform. Most preferably, the Fengycin B is the C₁₅ Fengycin B isoform. The Fengycin B, or active derivative thereof, may be acetylated.

In one embodiment, Fengycin B may have an amino acid sequence as set out in SEQ ID NO: 12:

[SEQ ID NO: 12]
L-Glu-D-Orn-D-Tyr-D-aThr-L-Glu-D-Val-L-Pro-L-
Gln-L-Tyr-L-ILe

In one embodiment where the peptide is a member of the Surfactin family, the general formula for the members of the Surfactin family may be set out in formula II below, wherein R1-R4 is any amino acid, and preferably R1 is glutamine or glutamic acid, R2 is leucine or valine, R3 is valine, leucine or alanine, and R4 is leucine or valine.

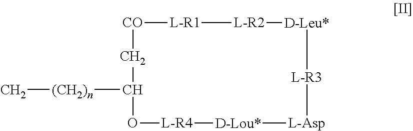

The member of the Surfactin family may be selected from a group consisting of: Esperin [SEQ ID NO: 15], Lichenysin [SEQ ID NO: 16], Pumilacidin [SEQ ID NO: 17] and Surfactin [SEQ ID NO: 18].

| | | |
|---|---|---|
| Esperin | L-Glu-L-Leu-D-Leu-L- [symbol] Asp-D-Leu-L-Leu-COOH | |
| [symbol] | L- [symbol] -L-Asp-D-Leu-L- [symbol] | [symbol] |
| [symbol] | L-Glu-L-Leu-D-Leu-L-Leu-L-Asp-D-Leu- [symbol] | |
| Surfactin | L-Glu-L- [symbol] -D-Leu- [symbol] -L-Asp-D-Leu [symbol] | [symbol] |

[symbol]

indicates data missing or illegible when filed

Wherein XL1 is Gln or Glu; XL2 is Leu or lie; XL4 and XL7 are Val or Ile XP7 is Val or Ile XS2 is Val, Leu or ILe; XS4 is Ala, Val, Leu or ILe; XS7 is Val Leu or lie.

Preferably, the member of the Surfactin family is Surfactin, or an active derivative thereof. In one embodiment, Surfactin may have an amino acid sequence as set out in SEQ ID NO: 18:

[SEQ ID NO: 18]
L-Glu-L-XS₂-D-Leu-L-XS₄-L-ASP-D-Leu-L-XS₇

Active derivatives of Surfactin may therefore comprise any of the C₁₂, C₁₃, C₁₄, C₁₅, C₁₆, or C₁₇ isoforms. Preferably, the Surfactin is the C₁₆ isoform. The Surfactin, or active derivative thereof, may be the C₁₂, C₁₃, C₁₄, C₁₅, C₁₆, or C₁₇ isoform. Most preferably, the Surfactin, or active derivative thereof, is the C₁₅ isoform.

In one embodiment, a Surfactin may have a structure as set out in formula III:

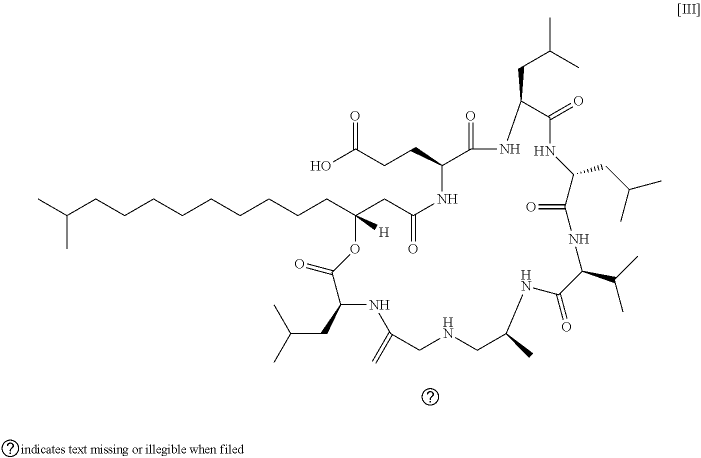 [SMILES: C=C1CNC[C@H](C)NC(=O)[C@H](C(C)C)NC(=O)[C@@H](CC(C)C)NC(=O)[C@H](CC(C)C)NC(=O)[C@H](CCC(=O)O)NC(=O)C[C@@H](CCCCCCCCCC(C)C)OC(=O)[C@H](CC(C)C)N1]

In one embodiment, the member of the Iturin family, or active derivative thereof may be selected from a group consisting of: Iturin A, Iturin A_{L}, Iturin C, Mycosubtilin, Bacillomycin D, Bacillomycin F, Bacillomycin L, Bacillomycin LC and Bacillopeptin.

In one embodiment, the general formula for members of the Iturin family or active derivative thereof may be as set out in formula IV below, wherein R1 to R5 is any amino acid, and preferably R1 is Asn or Asp, R2 is Pro, Gln or Ser, R3 is Glu, Pro or Gln, R4 is Ser or Asn and R5 is Thr, Ser or Asn:

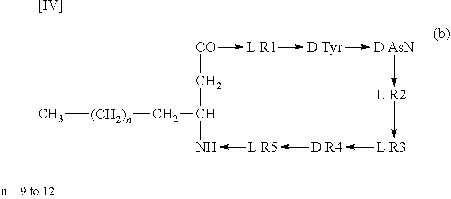

The member of the Iturin family, or active derivative thereof, may be Iturin A [SEQ ID NO:19], Iturin A_{L} [SEQ ID NO:20], Iturin C [SEQ ID NO:21], Mycosubtilin [SEQ ID NO:22], or Bacillomycin D [SEQ ID NO:23], Bacillomycin F [SEQ ID NO:24], Bacillomycin L [SEQ ID NO:25], Bacillomycin LC [SEQ ID NO:26], Bacillopeptin A, Bacillopeptin B or Bacillopeptin C [SEQ ID NO: 27] the sequences of which are shown below:

| | | |
|---|---|---|
| Bacillomycin [symbol] | L-Asn-D-Tyr-D-Asn-L-Pro-L-Glu-D-Ser-L-Thr | [symbol] |
| Bacillomycin [symbol] | L-Asn-D-Tyr-D-Asn-L-Gln-L-Pro-D-Asn-L-Thr | [symbol] |
| Bacillomycin [symbol] | L-Asn-D-Tyr-D-Asn-L-Ser-L-Gln-D-Ser-L-Thr | [symbol] |
| Bacillomycin [symbol] | L-Asn-D-Tyr-D-Asn-L-Ser-L-Glu-D-Ser-L-Thr | [symbol] |
| [symbol] | L-Asn-D-Tyr-D-Asn-L-Gln-L-Pro-D-Asn-L-Ser | [symbol] |
| [symbol] | L-Asn-D-Tyr-D-Asn-L-Gln-L-Pro-D-Asn-L-Ser | [symbol] |
| [symbol] | L-Asn-D-Tyr-D-Asn-L-Gln-L-Pro-D-Asn-L-Ser | [symbol] |
| [symbol] | L-Asn-D-Tyr-D-Asn-L-Gln-L-Pro-D-Ser-L-Asn | [symbol] |

[SEQ ID NO: 27]
Bacillopeptin A, B and C: cyclo [D-Asn-Ser-Glu-D-Ser-Thr-βAA-Asn-D-Tyr]

[symbol]

indicates data missing or illegible when filed

wherein the βAA for each of Bacillopeptin A, B and C is set out under R in Formula V below:

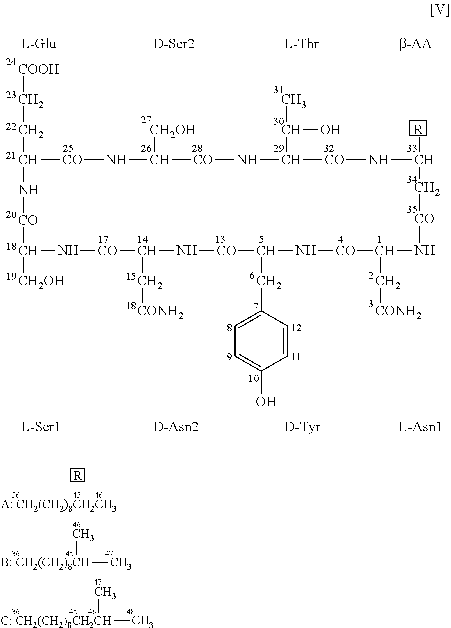

Preferably, the member of the Iturin family is Iturin A, or active derivative thereof. It will be appreciated that Iturin A is a lipopeptide. The Iturin A, or active derivative thereof, may be the C₁₄, C₁₅ or C₁₆ isoform. Active derivatives of Iturin A may therefore comprise any of the C₁₄, C₁₅ or C₁₆ isoforms. Most preferably, the Iturin A, or active derivative thereof, is the C₁₅ Iturin isoform.

In one embodiment, Iturin A may have an amino acid sequence as set out in SEQ ID NO: 19:

[SEQ ID NO: 19]
L-Asn-D-Tyr-D-Asn-L-Gln-L-Pro-D-Asn-L-Ser

Wherein n-C₁₄, i-C₁₅, ai-C₁₅

The skilled person would understand that the Chlorotetaine may also be referred to as Chlorotetain.

In one embodiment Chlorotetaine has a structure as set out in formula VI:

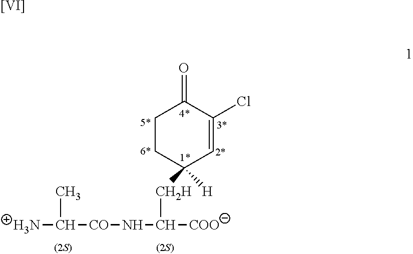

In another preferred embodiment, the microorganism of the invention may comprise a Malonyl CoA-acyl carrier protein transacylase gene from Bacillus amyloliquefaciens NCIMB 42971, which is provided herein as SEQ ID No: 7, as follows:

[SEQ ID No: 7]
ATGAACAATCTTGCCTTTTTATTTCCTGGACAAGGGTCTCAATTTGTAGGAATGGGCAAACAATTTTGGAATGATTTTGTGC
TCGCAAAGAGATTGTTTGAAGAAGCGAGCGATGCGATCTCCTTGGATGTAAAAAAACTGTGTTTTAACGGAGATATGAATGA
ATTGACAAAGACAATGAACGCGCAGCCCGCTATTTTAACGGTCAGTGTTATTGCTTTTCAAGTGTATATGCAGGAAATAGGG
GTGAAGCCCCGCTTCCTGGCAGGCCATAGCTTAGGCGAATATTCAGCGCTTGTCTGTGCCGGCGCCCTTTCTTTTCAGGATG
CCGTTACACTTGTAAGGCAGCGGGGAATTCTTATGCAGAATGCGGATCCTCAGCAGCAGGGGACGATGGCCGCCGTGACTCA
CCTCTCTCTTCAAACGTTGCAGGAAATATGTTCGAAAGTGTCGACGGAAGACTTTCCGGCAGGAGTAGCCTGCATGAATTCA
GAACAGCAGCATGTGATTTCCGGACACCGGCAAGCTGTGGAACGTGTCATCAAGATGGCGGAGGAAAAGGGAGCGGCATACA
CTTATTTGAATGTCAGTGCGCCTTTTCACAGTTCGCTGATACGATCAGCATCTGAACAATTCCAGACTGTATTACACCGGTA
TTCCTTCCGGGATGCCGCATGGCCGATCATTTCAAATGTCACCGCACGCCCTTACAGCAGCGGAAATTCAATCAGCGAACAT
CTCGAGCAGCACATGACGATGCCGGTAAGATGGACGGAATCGATGCATTACTTGCTTTTACACGGAGTCACAGAAGTCATCG
AAATGGGTCCGAACAATGTCTTAGCCGGTCTGCTGAGAAAAACAACGAATCACATTGTACCTTATCCCTTAGGACAGACATC
TGATGTTCACTTGCTTTCCAATTCAGCAGAAAGAAAGAAACATATTGTCCGTTTACGCAAAAAACAACTGAATAAATTGATG
ATTCAATCCGTCATTGCGCGAAATTACAACAAGGATTCAGCGGCTTATTCCAATATGACGACGGCATTATTTACGCAAATCC
AAGAGCTGAAAGAGAGAATGGAAAGACATGAAAATGAGCTCTCAGAACAAGAGCTCGAACATTCGATCCATTTATGCAAATT
AATTTGCGAGGCTAAACAGCTTCCGGATTGGGAAGAATTGCGGATTTTAAAATAA

Accordingly, preferably the microorganism comprises a nucleotide sequence substantially as set out in SEQ ID No: 7, or a variant or fragment thereof.

In one embodiment, the Malonyl CoA-acyl carrier protein transacylase gene from Bacillus amyloliquefaciens NCIMB 42971 may encode an amino acid sequence provided herein as SEQ ID No: 9, as follows:

[SEQ ID No: 9]
MNNLAFLFPGQGSQFVGMGKQFWNDFVLAKRLFEEASDAISLDVKKLCFNGDMNELTKTMNAQPATLTVSVIAFQVYMQEIG
VKPRFLAGHSLGEYSALVCAGALSFQDAVTLVRQRGILMQNADPQQQGTMAAVTHLSLQTLQEICSKVSTEDFPAGVACMNS
EQQHVISGHRQAVERVIKMAEEKGAAYTYLNVSAPFHSSLIRSASEQFQTVLHRYSFRDAAWPIISNVTARPYSSGNSISEH
LEQHMTMPVRWTESMHYLLLHGVTEVIEMGPNNVLAGLLRKTTNHIVPYPLGQTSDVHLLSNSAERKKHIVRLRKKQLNKLM
IQSVIARNYNKDSAAYSNMTTALFTQIQELKERMERHENELSEQELEHSIHLCKLIC EAKQLPDWEELRILK

Accordingly, preferably the microorganism comprises a gene encoding the amino acid sequence substantially as set out in SEQ ID No: 9, or a variant or fragment thereof.

In another preferred embodiment, the microorganism of the invention may comprise a Malonyl CoA-acyl carrier protein transacylase gene from Bacillus amyloliquefaciens NCIMB 42971, which is provided herein as SEQ ID No: 8, as follows:

[SEQ ID No: 8]
ATGTATACCAGTCAATTCCAAACCTTAGTAGATGTCATTCGGGAAAGAAGCAATATCTCTGACCGCGGGATCCGTTTTATCGAATCCGATA
AAAACGAGACGGTTGTCTCTTATCGCCAATTGTTTGAAGAGGCGCAAGGGTATCTTGGCTATTTACAGCATATCGGCATTCAGCCGAAGCA
GGAAATTGTATTTCAAATCCAAGAAAACAAATCATTTGTCGTTGCTTTTTGGGCTTGTATATTAGGAGGAATGATCCCGGTGCCGGTCAGT
ATCGGAGAAGATGATGACCATAAGCTGAAGGTCTGGCGCATTTGGAATATATTAAATCACCCGTTTCTGATTGCCTCTGAAAAAGTATTGG
ACAAAATAAAGAAATACGCTGCAGAACACGATTTACAGGATTTCCATCATCAATTAAACGAAAAATCTGACATCATTCAAGATCAAACCTA
CGATTACCCCGCTTCGTTTTATGAACCTGATGCGGATGAACTCGCCTTTATCCAATTTTCTTCAGGATCGACAGGAGATCCAAAAGGAG
TCATGTTAACGCATCACAACTTAATACATAACACGTGCGCCATTGGGAATGCCCTAGCCGTTCATTCGAGAGACTCTTTCTTATCATG
GATGCCTTTAACGCATGATATGGGGCTCATCGCCTGCCACCTTGTTCCCTTCATAACCGGAATCAATCAAAATCTGATGCCTACAGAATTA
TTTATTCGCAGACCTATTCTTTGGATGAAAAAAGCTCATGAACATAAAGCCAGTATTCTATCCTCTCCTAATTTCGGATACAACTACTTCC
TTAAATTTCTGAAAAACGAACCAGACTGGGATTTATCCCACATCAAGGTCATCGCAAACGGTGCAGAACCGATATTGCCGGAGCTCTGTGA
CGAATTTTTGAAAAGATGCGCAGCATTCAATCTGAAAAGATCCGCCATTTTGAATGTTTACGGTTTAGCGGAAGCTTCGGTCGGCGCAGCA
TTCTCTAAATTAGGTAAAGAATTCGTTCCCGTTTATTTGCATCGCGATCATTTAAATCTCGGTGAAAGAGCTGTAAACGTCAGCAAAGAGG
ATCAAAATTGCGCTTCATTCGTCGAAGTGGGACGACCTATTGACTATTGTCAGCTTCGGATCTCCGATGAAGCAAATGAAAGAGTAGA
AGACGGAATCATCGGCCATATCCAGATCAAAGGAGACAATGTGACTCAAGGGTATTATAACAACCCCGAGAGTACGGAAAAAGCGCTGA
CTTCTGACGGCTGGGTAAAAACGGGAGACCTCGGATTCATTAGTGAAAGTGGTAACTTAGTCGTAACCGGAAGAGAAAAGGACATTATTTT
CGTGAACGGAAAAAATATCTACCCGCACGATATTGAACGGGTAGCGATTGAAATGGAAGAGGTTGACTTAGGAAGGGTTGCCGCCTGCGGT
GTATATGATCAAAAGACACAAAGCGGAGAAATCGTGCTCTTTGTTGTTTACAAAAAATCACCTGAAAAATTCGCACCGCTTGTCAAAGAGA
TAAAAAAGCATTTGCTCAAGCGGGGCGGCTGGAGCATAAAAGATGTCCTTCCGATCCGAAAACTCCCTAAAACAACCAGCGGAAAGGTTAA
ACGCTACGAACTTGCCAGACAGTATGAGGCAGGGAATTTTTCAACAGAGTCTGCCGCCATCAATGAATATTTGGAGAGCAGCCCGGAAACG
TCCGGACAGACTCCCATTCATGAAATTGAAACGGAATTACTGTCTATCTTTTCCGATGTGCTCAATGGGAAAAAGGTTCACCTCGC
TGACAGTTATTTTGATATGGGAGCAAATTCATTACAGTTATCGCAGATTGCCGAGCGCATAGAACAGAAATTCGGACGCGAGCTTGCCGTT
TCAGATCTCTTTACGTATCCTTCTATCACTGATTTAGCGGCGTATCTGTCTGAAAGCCGGGCTGAAATCAAGCAGGACGTGGCAGCTAAAC
CAAGCCATGTGACACCGAAAGATATCGCCATTATCGGGATGTCGCTCAATGTCCCTGGAGCATCAACTAAAAATGATTTTTGGAATCTGCT
TGAAAAAGGTGAGCACAGCATTCGAGAATACCCTGCATCCCGGCTGAAAGATGCGGCGGATTATTTAAAGTCCATCCAAAGCGAAATCAAT
GAGAATCAGTTTGTGAAGGGCGGCTATTTAGATGAAATCGACCGCTTTGATTTCTCGTTCTTCGGTTTAGCTCCTAAAACGGCTCAGTTTA
TGGACCCTAACCAAAGACTGTTTTTGCAGTCTGCATGGCATGCGATTGAAGATGCGGGCTATGCCGGCGGCAGCATGAACGGGAGCCGT
GTCGGGGTATATGCAGGGTACTCGAAGGTGGGCTACGATTATGAACGTCTCCTTTCTGCGAATTATCCGGAGGAGCTTCATCAATATA
TCGTGGGCAATCTCCCTTCCGTGTTAGCCAGCCGAATCGCTTATTTCTTAAATTTAAAAGGGCCGGCGGTCACAGTCGATACGGCGTGCTC
CTCATCGCTTGCCGCCGTTCATATGGCATGTAAATCTTTAATATCCGGCGATTGTGAAATGGCTCTTGCCGGCGGTATCCGGACATCGCTC
TTGCCGATCTGTATCGGACTTGATATGGAATCTTCGGACGGGTACACGAAAACGTTCAGCAAAGATTCAGACGGTACTGGCACAGGTGAAG
GCGCGGCCGCAGTCCTGCTGAAACCTCTGCAGGATGCTGTTCGCGACGGAGACCATATTTACGGCGTAATCAAGGGAAGCGCGTTGAATCA
AGACGGAACAACCGCCGGGATTACAGCACCGAATCCGGCAGCTCAGACTGAGGTCATTGAGACGGCCTGGAAAGACGCGGGCATTGCCCCT
GAAACACTGTCTTTCATCGAAGCGCATGGCACCGGAACGAAGCTCGGCGATCCGGTTGAATTTAACGGGCTTTGTAAAGCGTTTGAA
AAGTATACGGCAAAAAAACAATTTTGTGCGATTGGTTCTGTTAAATCGAACATCGGTCATTTGTTTGAAGCGGCAGGCATCGTCGGGCTG
ATCAAATCTGTCCTCATGCTGAATCACAAGAAAAATCCGCCGTTAGTGCACTTTAATGAACCTAATCCGCTCATTCATTTTCACTCTTCAC
CATTTTACGTAAACCAGGAAGCTGCAGCGTTCCCATCCGGTGATGAGCCGCTGCGAGGCGGAGTCAGCTCATTTGGCTTTAGCGGAACGAA
CGCTCATGTGGTATTGGAAGAATATATTTCTCAAAGTGAGTATGCGCCCGAGGATGAACATGGGCCGCACCTATTTGTTTTATCCGCTCAT
ACTGAAAAATCACTCTATGAACTCGCACAGCAGTACCGGCAATATGTATCGGATGACAGCCAAGCTTCATTAAAGTCCATTTGCTATACAG
CCAGTACGGGCAGGGCTCATTTGGATCATGGCATTGCCATGATTGTATCCGGTAAACAAGAACTATCGGATAAGCTGACCCGCCTGATTC
AGGGAGACAGAAACCTTCCCGGTGTATACATCGGCTACAAGAATATGAAGGAAATGCTGCCCGCTCATAAAGAAGAGCTGAATAAAC
AAGCAGCCGCACTGATTAAGCAGCGTTTACGTACGCAAGATGAACGGATCACATGGCTGCATCGCGCCGCCGAATTATTTGTGCAAGGAGC
CGTTATCGATTGGCGCGCGCTTTATTCAGGTGAAACTGTACAAAAGACGCCATTGCCCTTGTATCCGTTTGAACGGAGCCGATGCTGGGCT
GAAGCTGACCAATTGCGCTTAAACGAGGACGAAAAGAGAGGAGAAGCGGCATTGAATATCAATCAATCGAAGTCGCATATTGAATCCTTCC
TGAAAACTGTAATCAGCAATACTTCGGGGATCAGAGCGGAGGAACTCGATCTGAATGCTCATTTTATCGGACTCGGAATGGATTCTATCAT
GCTGTCACAGGTCAAAAAAGCCATCGCGGACGAATTTGGGGCAGACATCCCGATGGATCGTTTTTTTGATACGATGAACAACCTTCAAAGT
GTCATAGATTACTTGGCTGAGACCGTTCCAACGTCCTTTGCATCCGCTCCGCCTCAAGAGAATGTTCCGGCGCAGGAAATGCAGGTCAT
TTCAGAAGCACAGTCTGAATCGGATCGCAGAGAAGGTCATCAAGAGCATATGCTCGAAAAAATAATCGCTTCTCAGAATCAATTAATT
CAGGATACCTTGCAAGCTCAATTAAATAGCTTTAATTTGTTGAGAAACAGCGGACATCATTCCGATGAGAAAGAATACGCTAAAGCGCAAG
AGAGATCAATTCCTTCTGTCCAGCAGGGGCCTCCGGCCGTCACTGCAGAAAAGAAAGCGGCTCAAGAAGCGAAACCCTATGTTCCTTTCCA
GCCTCAGAACCTGCATGAACAGGGACACTATACCGCACGGCAAAAACAATACTTAGAAGATTTCATCAAGAAATACGCAGATAAAACGAAA
GGTTCCAAACAATATACGGACAACACCCGATTTGCTCATGCAAACAACCGCAACTTGTCCAGCTTCCGTTCATATTGGAAGGAAATCGTAT
ACCCGATTATCGCCGAACGTTCTGACGGTTCTAAAATGTGGGATATTGACGGAAATGAATATATCGATGTCACCATGGGATTCGGGGTTAA
CCTTTTCGGGCATCATCCTTCCTTTATTACACAGGTTATCGATGATTCAGCCCGTTCTTCATTGCCTCCGCTCGGACCGATGTCAGA
TGTCGCCGGTGAAGTTGCCGACCGGATCCGCACATGTACCGGGGTAGAAAGGGTCGCTTTCTATAATTCCGGAACAGAGGCCGTCATGGT
TGCCCTGCGTTTGGCGCGGGCGGCAACAGGAAGAAAGAAAGTGGTGGCGTTCTCGGGCTCTTATCACGGCACGTTTGACGGCGTATTAGGG
GTTGCCGGCACAAAAGGCGGAGCTGCGTCTGCGAATCCGCTGGCTCCTGGTATACTGCAGAGCTTTATGGATGATTTGATTATTTTACATT
ACAACAATCCCGATTCTCTGGACGTGATCCGCAGTCTTGGTGATGAATTGGCAGCCGTACTGGTGGAACCGGTACAAAGCCGCAGACCGGA
TTTGCAGCCGCGGGCATTTTTGAAAGAATTGCGGGCGATCACGCAGCAATCCGGAACAGCTCTGATTATGGATGAAATTATTACCGGATTT
CGGATCGGTCTCGGCGGCGCACAGGAATGGTTCGGCATTCAGGCTGATTTAGTGACCTACGGAAAAATCATCGGCGGCGGACAGCCGTTA
GGGGTAGTTGCCGGAAAAGCTGAGTTCATGAATGCGATCGACGGGGGTACCTGGCAGTATGGGGACGATTCCTACCCGCAAGACGAG
GCGAAACGCACGTTTGTGGCCGGAACCTTCAATACTCATCCGCTTACCATGAGAATGTCATTAGCCGTGCTTCGTCATTTACAAACCGAGG
GAGAACATCTGTATGAGCAGCTTAATCAAAAAACAGCCTACTTGGTGGATGAGCTGAATCGCTGCTTCGAACAAGCGCAAGTGCCTATCCG
CATGGTTCGATTCGGTTCTTTATTCCGGTTTGTCTCATCGCTTGATAATGACTTGTTCTTTTACCATCTCAACTATAAAGGTGTCTATGTG
TGGGAAGGACGCAACTGCTTCTTGTCTGCGGCGCATACCGCTGATGATATCGAAAAGATTATTCAAGCGGTGAAAGACACGGTGGAGGATC
TTCGCCGAGGCGGATTTATTCCGGAAGGCCCGGACTCCCCTGATGGCGGAGGCCGTAAAAAGTCCGGGACGCGCGAGCTTTCACCTGAACA
AAAGCAGTTGGTTATGGCATCCCATTACGGGAATGAAGCGTCCGCCGCTTTAAACCAGTCCATTATGCTGAAAGTGGAGGGCGAACTGCAG
CATACACCATTAAAACAAGCCGTCCGGCATATCGTTGGCCGTCATGAAGCTTTACGTACGGTGATTCATCCCGATGACGAGGTACAGCAAG
TGCAGGAACGGATGAATATAGAAATACCAGTCATTGATTTTACCGTTCACCCGCATGAACATCGGGAGTCGGAAATTCAAAAATGGCTGAC
AGAAGATGCCAAGCGGCCGTTCCATTTCCATGAACAAAAGCCTTTGTTTAGAATCCATGTGCTTACATCGGCTCACAATGAACATCTGATT
GTGCTCACGTTCCATCATATCATTGCCGATGGATGGTCAATCGCCGTATTTGTTCAAGAACTGGAGAGCAACTACGCGGCAATCGTACAAG
GAAAACCGATTTCACCGAAAGAGGCAGATGTTTCGTTTCGCCAATACTTAGACTGGCAGCAGGCACAGATTGACAGCGGCCATTATGAAGA
AGGGGTCCGTTATTGGCGGCGTCATTTCTCTGAACCGATTCAGCAGCCAATTCTGCCGAGCACAGGTTCTGTCCGTTATCCGAACGGGTAT
GAGGGAGACCGGTGTACCGTCAGGCTTGGACGGCCATTGAGCGAGGCTTTAAGGTCATTAAGCATTCAGATGAAAAATAGCGTATTTGTGA
CAATGCTGGGTGCATTTCATCTTTTTCTGCACCGGCTTACCAAACAGTCAGGCCTTGTGATCGGGATCCCCGCAGCAGGTCAATCGCATAT
AAAACAGCATGATCTGATTGGAAATTGCGTCAATATGATTCCGGTGAAGAACACGTCTACTTCAGAAAGCACTTTAACCGGTTATCTTGGC
AGTATGAAAGAAAGCGTGAATCTTGCAATGCGGCACCAAGCCGTCCCGATGACACTGGTGGCCAGAGAGCTTCCGCACGATCAAGTGCCGG
ATATGCGTATTATCTTTAATTTAGACAGGCCTTTTCGAAAGCTGCATTTCGGAAAGGCGGAAGCGGAGCCCGTTGCATACCCGGTAAAATG
CACCCTGTACGATTTATTTCTTAACATAACAGACGCGCATCAAGAATATGTTCTTGATTTCGACTTTAATACGAACGTCATCAGTCCGGAA
ATCATGAAAAAGTGGGGAGCGGGTTTTACAAATTTGCTGCAAAAAATGGTTGAGGGGGATTCAATCCCTCTTGACGCCATGATGATGTTTT
CCGATGAAGAACAGCATGATTTACAAAAACTGTATGCCGAACACCAGAAGCGGGTCTCTTCAATAGGAAGCAATACAGCAAATTTCACTGA
AGCCTACGAGGCGCCGATAAATGAAACGGAACGGCAGCTGGCGCGGATTTGGGAGGAACTTTTCGGCCTTGAACGGGTCGGCAGATCAGAT
CGCTTTCTGGCTCTGGGAGGAAACTCGCTCCAGGCGACGCTTATGCTTTCCAAAATTCAGAAGACATTTCATCAAAAGGTTTCCATCGGAC
AATTTTTCAATCACCAGACTGTTAAGGAATTAGCACATTTCATTCAGAACGAAACAAAAGTCGTGCACCTCCCGATGAAAGCTGCCGAGAA
AAAAGCGTATTACCCGACATCGCCGGCGCAGCAAAGAGTATATTTCCTGCACCAACTGGAACCGGATCAGCTGGCGCAAAATATGTTCGGC
CAAATATCAATAACAGGGAAGTACGATGAGCAAGCCCTGATCTCATCTCTTCAACAAGTGATGCAGCGGCACGAAGCGTTTCGCACGTATT
TCGACATTATAGATGGCGATATCGTTCAGAAACTTGAAAACGAAGTTGATTTTAACGTTCATGTCCGGACAATGAGCCGGGACGAATTTGA
TGCCTATTCAGACCGGTTTGTAAAACCGTTCCGCCTGGACCAAGCTCCGTTAGTTCGTGCGGAGCTGATCAAGATTGAAAACGAGCAGGCC
GAACTGCTCATCGATATGCATCATATCATTTCGGATGGTTATTCCGTCAACATCCTTACAAATGAATTGCTGGCTTTATATCATCAGAAAC
CATTACCGGACATTGAATTTGAATATAAAGATTTCGCAGAATGGCAAAACCAACGGCTGAATGAGGATGCCATGAAGCGGCAGGAGACATA
TTGGCTGGAACAATTTCAAGACGAAATTCCCATCCTTGACCTGCCGACAGACGGTTCAAAAGCGGCAGAACGGTCTTCTGAGGGACAGCGT
GTGACATGCTCCTTACAGCCGGATGTCATCCGTTCGCTTCAAGATTTGGCGCAAAAGGCGGAAACCACTCTCTATACGGTGCTTCTGGCCG
CCTATAATGTGCTGCTTCATAAATATACCGGACAAGAAGACATTGTCGTAGGCACGCCTGCTTCAGGAAGAAATCATCCGGATATCGAAAA
AATCATCGGTATTTTCATACAAACCATCGGAATCCGGACGAAGCCGCACGCCAATAGAACGTTTACGGATTATCTGGAAGAAGTAAAGCGG
CAGACGCTTGACGCTTTCGAAAACCAAGACTATCCATTCGACCGGCTTGTGGAGAAATTAAATGTGCAGCGGGAAACAACCGGAAAGTCT
CTGTTTAACACGATGTTTGTGTTTCAAAACATTGAATTTCATGAAATCCGGCACAATGAATGTACATTTAAAGTGAAAGAACGAAAT
CCAGGGGTCTCTTTGTATGATTTGATGCTCACGATCGAAGATGCCGGTCAACAGATAGAGATGCATTTTGATTATAAACCGGGACGATTCA
CAAAAGACACCATTGAACAGATCACCAGACACTATACCGGCATTTTAAACAGTCTTGTTGAGCAGCCGGAGATGACATTGTCTTCCGTTCC
TATGCTGTCTGAAACCGAACGGCATCAACTGCTGACGGAGTGTAACGGCACAAAGACGCCGTATCCGCATAACGAAACAGTAACCCGATGG
TTTGAAATGCAGGCGGAACAGAGTCCCGATCATGCAGCCGTTATTTTTGGCAATGAGCGGTATACGTACAGACAGCTCAATGAACGGGCGA
ACCGATTGGCGCGGACGTTACGGACAAAAGGCGTACAAGCGGATCAATTCGTTGCCATCATCTCTCCGCATCGCATCGAGTTGATTGTTGG
TATTTTGGCTGTTCTGAAATCAGGCGGCGCATACGTGCCTATTGATCCTGAATATCCGGAAGATCGGATCCAATATATGCTGAGAGATTCA
AGGGCGGAGGTTGTGTTGACACAGCGCAGCCTGCTGGATCAATTACCGTATGATGGTGACGTTGTGCTTTTGGATGAGGAAAACTCATACC
ATGAGGATCACTCGAATCTTGAATCGGACAGCGATGCGCATGATTTGGCCTACATGATCTATACGTCAGGTTCCACGGGAAATCCAAAAGG
TGTCCTCATTGAGCATCAGGGACTGGCTGATTATATTTGGTGGGCGAAAGAGGTTTATGTAAGAGGTGAGAAAACCAACTTCCCATTATAC
TCTTCCATCTCTTTCGATCTGACTGTGACCTCGATATTTACACCGCTGGTTACGGGAAATACCATCATTGTCTTTGACGGCGAAGACAAAA
GCGCTGTGCTTTCTGAGATTATGCGGGACTCAAGAATAGACATGATCAAATTGACCCCGGCACATCTGCACGTCATCAAGGAGATGAATAT
CGGTGGCGGCACCGCAATACGGAAAATGATTGTCGGCGGAGAAAATTTAAGCACCCGTCTGGCCAAAAGTGTCAGCGAGCAGTTTAAAGGC
CGGCTGGACATTTTCAATGAATACGGACCGACGGAAGCTGTCGTCGGATGTATGATTTATCACTTCGACGCAGAACGGGACAAGCGGGAAT
TTGTACCGATCGGCACTCCGGCTGCCAACACGGATATTTATGTGGCCGATGCAAGCAGAAATCTGGTTCCGATCGGGGTAATCGGCGAAAT
ATATATCAGCGGACCGGGTGTTGCCAGAGGGTATTGGAACCGTCCGGATTTAACGGCAGAGAAATTTGTTGAAAACCCGTATGTCCCGGGA
GCGAAGATGTACAAATCAGGGGATTTGGCTAAGCGGTTGAAGGACGGAAACCTTGTATATATCGGGCGCGTTGATGAACAAGTCAAAATCA
GGGGATACCGAATCGAGCTTGGTGAAATTGAAGCAGCAATGCATAACGCGGAAGCGGTGCAAAAAGCCGCGGTTACAGTGAAAGAAGAAGA
AGACGGCTTAAAACAATTATGCGCGTATTACGTAAGCGACAAGCCTATAGCGGCTGCGCAGCTTAGGGAACAATTGTCATCGGAGCTTCCG
GACTACATGGTTCCGTCCTATTTTGTCCAACTGGAGCATATGCCGTTAACGTCCAACGGGAAAATAAACCGTAAGGCACTGCCAGCACCAG
AAGCGAGTCTGCAGCAGACAGCTGAATATGTTCCGCCGGGTAATGAGACGGAGTCCAAACTGACAGATTTATGGAAGGAAGTGCTCGGAAT
AAGCCATGCGGGGATCAAACATAATTTCTTTGATCTCGGAGGCAACTCCATCCGAGCGGCTGCCTTGGCCGCCAGAATTCACAAAGAGCTG
GATGTGAATCTGTCTCTCAAAGACATATTCAAGTTTCCTACCATTGAACAATTGGCTGACAAGGCGTTACACATGGACAAAAACCGATATG
TACCGATTCCGGCTGCAAAGGAAATGCCATATTATCCGGTTTCTTCAGCTCAGAGGCGTATGTATTTGTTAAGTCACACAGAAGGCGGCGA
GCTGACTTACAATATGACGGGTGCCATGAATGTGGAAGGGACGATCGATCCCGAACGGTTAAACGCCGCTTTCCGAAAATTAATCGCGCGT
CATGAAGCGTTGCGGACCAGCTTTGATTTATATGAAGGCGAGCCGGCACAGCGTATTCATCAGAACGTCGACTTTACGATAGAACGGATTC
AAGCAAGCGAAGAAGAAGCGGAAGACCGTGTGCTTGATTTCATCAAAGCGTTTGACTTAGCCAAACCGCCGCTGATGCGGGCCGGACTGAT
TGAAATTGAACCTGCGCGGCACGTGCTTGTGGTTGATATGCATCATATCATTTCTGACGGCGTGTCCGTCAATATTCTGATGAAAGATTTA
AGCCGAATCTACGAGGGGAACGAACCGGACCCGCTCTCTATTCAATATAAAGACTTTGCAGTTTGGCAGCAATCGGACATTCAGAAACGGA
ACATCAAGAGCCAGGAAGCGTATTGGCTGGATCAGTTTCACAGTGATATTCCTGTACTGGATATGCCTGCGGATTATGAGAGACCTGCCAT
ACGCGATTACGAAGGCGAATCATTTGAATTTCTTATACCCGAACACTTGAAACAGCGTTTAAGCCAAATGGAAGAAGACACAGGAGCAACA
CTGTATATGATTTTATTGGCGGCCTATACGATTCTTTTATCCAGGTACAGCGGACAAGAAGATATTATCGTAGGAACGCCATCTGCCGGGC
GGACTCATTTGGATGTAGAGCCGGTCGTGGGAATGTTCGTCAATACGTTAGTCATTCGCAATCACCCGGCGGGCCGTAAAACATTTGATGC
CTACTTAAACGAAGTAAAAGAAAACATGCTGAACGCCTATAAAAATCAAGACTATCCATTGGAAGAATTAATTCAGCATCTGCATCTTCCA
AAAGATTCAAGCCGCAATCCTTTATTCGATACGATGTTTGTGCTGCAAAATCTCGATCATGCTGAATTGACTTTCGAT
TCTCTTCAACTCAAGCCGTATTCATTTCATCATCCGGTTGCCAAATTCGATTTGACCTTGTCGATTCAGGCGGACCAAGACAACTA
TCACGGACTGTTTGAATATTCGAAAAAACTGTTTAAGAAAAGCAGAATCGAGGTTTTATCAAACGACTACTTACACATTCTATCGGCGATT
TTGGAACAACCAAGCATTCTAATTGAACATATCGGATTGAGCGGCAGCAATGAGGAAGAAGAGAACGCGCTTGATTCTA
TTCAATTGAACTTTTAG

Accordingly, preferably the microorganism comprises the nucleotide sequence substantially as set out in SEQ ID No: 8, or a variant or fragment thereof.

Therefore, the microorganism may comprise a gene with at least 90% sequence identity to SEQ ID NO: 7 or at least 90% sequence identity to SEQ ID NO: 8, such as at least 95% sequence identity to SEQ ID NO: 7, at least 95% sequence identity to SEQ ID NO: 8, at least 98% sequence identity to SEQ ID NO: 7, at least 98% sequence identity to SEQ ID NO: 8, at least 99% sequence identity to SEQ ID NO: 7, at least 99% sequence identity to SEQ ID NO: 8, 100% sequence identity to SEQ ID NO: 7 or 100% sequence identity to SEQ ID NO: 8.

In one embodiment, Malonyl CoA-acyl carrier protein transacylase gene from Bacillus amyloliquefaciens NCIMB 42971 may encode an amino acid sequence provided herein as SEQ ID No: 10, as follows:

[SEQ ID No: 10]
MYTSQFQTLVDVIRERSNISDRGIRFIESDKNETVVSYRQLFEEAQGYLGYLQHIGIQPKQEIVFQIQENKSFVVAFWACILGGMIPVPVS
IGEDDDHKLKVWRIWNILNHPFLIASEKVLDKIKKYAAEHDLQDFHHQLNEKSDIIQDQTYDYPASFYEPDADELAFIQFSSGSTGDPKGV
MLTHHNLIHNTCAIGNALAVHSRDSFLSWMPLTHDMGLIACHLVPFITGINQNLMPTELFIRRPILWMKKAHEHKASILSSPNEGYNYFLK
FLKNEPDWDLSHIKVIANGAEPILPELCDEFLKRCAAFNLKRSAILNVYGLAEASVGAAFSKLGKEFVPVYLHRDHLNLGERAVNVSKEDQ
NCASFVEVGRPIDYCQLRISDEANERVEDGIIGHIQIKGDNVTQGYYNNPESTEKALTSDGWVKTGDLGFISESGNLVVTGREKDIIFVNG
KNIYPHDIERVAIEMEEVDLGRVAACGVYDQKTQSGEIVLFVVYKKSPEKFAPLVKEIKKHLLKRGGWSIKDVLPIRKLPKTTSGKVKRYE
LARQYEAGNFSTESAAINEYLESSPETSGQTPIHEIETELLSIFSDVLNGKKVHLADSYFDMGANSLQLSQIAERIEQKFGRELAVSDLFT
YPSITDLAAYLSESRAEIKQDVAAKPSHVTPKDIAIIGMSLNVPGASTKNDFWNLLEKGEHSIREYPASRLKDAADYLKSIQSEINENQFV
KGGYLDEIDRFDFSFFGLAPKTAQFMDPNQRLFLQSAWHAIEDAGYAGGSMNGSRVGVYAGYSKVGYDYERLLSANYPEELHQYIVGNLPS
VLASRIAYFLNLKGPAVTVDTACSSSLAAVHMACKSLISGDCEMALAGGIRTSLLPICIGLDMESSDGYTKTFSKDSDGTGTGEGAAAVLL
KPLQDAVRDGDHIYGVIKGSALNQDGTTAGITAPNPAAQTEVIETAWKDAGIAPETLSFIEAHGTGTKLGDPVEFNGLCKAFEKYTAKKQF
CAIGSVKSNIGHLFEAAGIVGLIKSVLMLNHKKNPPLVHFNEPNPLIHFHSSPFYVNQEAAAFPSGDEPLRGGVSSFGFSGTNAHVVLEEY
ISQSEYAPEDEHGPHLFVLSAHTEKSLYELAQQYRQYVSDDSQASLKSICYTASTGRAHLDHGIAMIVSGKQELSDKLTRLIQGDRNLPGV
YIGYKNMKEMLPAHKEELNKQAAALIKQRLRTQDERITWLHRAAELFVQGAVIDWRALYSGETVQKTPLPLYPFERSRCWAEADQLRLNED
EKRGEAALNINQSKSHIESFLKTVISNTSGIRAEELDLNAHFIGLGMDSIMLSQVKKAIADEFGADIPMDRFFDTMNNLQSVIDYLAETVP
TSFASAPPQENVPAQEMQVISEAQSESDRREGHQEHMLEKIIASQNQLIQDTLQAQLNSFNLLRNSGHHSDEKEYAKAQERSIPSVQQGPP
AVTAEKKAAQEAKPYVPFQPQNLHEQGHYTARQKQYLEDFIKKYADKTKGSKQYTDNTRFAHANNRNLSSFRSYWKEIVYPIIAERSDGSK
MWDIDGNEYIDVTMGEGVNLFGHHPSFITQVIDDSARSSLPPLGPMSDVAGEVADRIRTCTGVERVAFYNSGTEAVMVALRLARAATGRKK
VVAFSGSYHGTFDGVLGVAGTKGGAASANPLAPGILQSFMDDLIILHYNNPDSLDVIRSLGDELAAVLVEPVQSRRPDLQPRAFLKELRAI
TQQSGTALIMDEIITGFRIGLGGAQEWFGIQADLVTYGKIIGGGQPLGVVAGKAEFMNAIDGGTWQYGDDSYPQDEAKRTFVAGTENTHPL
TMRMSLAVLRHLQTEGEHLYEQLNQKTAYLVDELNRCFEQAQVPIRMVRFGSLFREVSSLDNDLFFYHLNYKGVYVWEGRNCFLSAAHTAD
DIEKIIQAVKDTVEDLRRGGFIPEGPDSPDGGGRKKSGTRELSPEQKQLVMASHYGNEASAALNQSIMLKVEGELQHTPLKQAVRHIVGRH
EALRTVIHPDDEVQQVQERMNIEIPVIDFTVHPHEHRESEIQKWLTEDAKRPFHFHEQKPLFRIHVLTSAHNEHLIVLTFHHIIADGWSIA
VFVQELESNYAAIVQGKPISPKEADVSFRQYLDWQQAQIDSGHYEEGVRYWRRHFSEPIQQPILPSTGSVRYPNGYEGDRCTVRLGRPLSE
ALRSLSIQMKNSVFVTMLGAFHLFLHRLTKQSGLVIGIPAAGQSHIKQHDLIGNCVNMIPVKNTSTSESTLTGYLGSMKESVNLAMRHQAV
PMTLVARELPHDQVPDMRIIFNLDRPFRKLHEGKAEAEPVAYPVKCTLYDLFLNITDAHQEYVLDFDENTNVISPEIMKKWGAGFTNLLQK
MVEGDSIPLDAMMMESDEEQHDLQKLYAEHQKRVSSIGSNTANFTEAYEAPINETERQLARIWEELFGLERVGRSDRFLALGGNSLQATLM
LSKIQKTFHQKVSIGQFFNHQTVKELAHFIQNETKVVHLPMKAAEKKAYYPTSPAQQRVYFLHQLEPDQLAQNMFGQISITGKYDEQALIS
SLQQVMQRHEAFRTYFDIIDGDIVQKLENEVDFNVHVRTMSRDEFDAYSDRFVKPFRLDQAPLVRAELIKIENEQAELLIDMHHIISDGYS
VNILTNELLALYHQKPLPDIEFEYKDFAEWQNQRLNEDAMKRQETYWLEQFQDEIPILDLPTDGSKAAERSSEGQRVTCSLQPDVIRSLQD
LAQKAETTLYTVLLAAYNVLLHKYTGQEDIVVGTPASGRNHPDIEKIIGIFIQTIGIRTKPHANRTFTDYLEEVKRQTLDAFENQDYPFDR
LVEKLNVQRETTGKSLENTMFVFQNIEFHEIRHNECTFKVKERNPGVSLYDLMLTIEDAGQQIEMHEDYKPGRETKDTIEQITRHYTGILN
SLVEQPEMTLSSVPMLSETERHQLLTECNGTKTPYPHNETVTRWEEMQAEQSPDHAAVIFGNERYTYRQLNERANRLARTLRTKGVQADQF
VAIISPHRIELIVGILAVLKSGGAYVPIDPEYPEDRIQYMLRDSRAEVVLTQRSLLDQLPYDGDVVLLDEENSYHEDHSNLESDSDAHDLA
YMIYTSGSTGNPKGVLIEHQGLADYINWAKEVYVRGEKTNFPLYSSISFDLTVISIFTPLVTGNTIIVFDGEDKSAVLSEIMRDSRIDMIK
LTPAHLHVIKEMNIGGGTAIRKMIVGGENLSTRLAKSVSEQFKGRLDIFNEYGPTEAVVGCMIYHFDAERDKREFVPIGTPAANTDIYVAD
ASRNLVPIGVIGEIYISGPGVARGYWNRPDLTAEKFVENPYVPGAKMYKSGDLAKRLKDGNLVYIGRVDEQVKIRGYRIELGEIEAAMHNA
EAVQKAAVTVKEEEDGLKQLCAYYVSDKPIAAAQLREQLSSELPDYMVPSYFVQLEHMPLTSNGKINRKALPAPEASLQQTAEYVPPGNET
ESKLTDLWKEVLGISHAGIKHNFFDLGGNSIRAAALAARIHKELDVNLSLKDIFKETTIEQLADKALHMDKNRYVPIPAAKEMPYYPVSSA
QRRMYLLSHTEGGELTYNMTGAMNVEGTIDPERLNAAFRKLIARHEALRTSFDLYEGEPAQRIHQNVDFTIERIQASEEEAEDRVLDFIKA
FDLAKPPLMRAGLIEIEPARHVLVVDMHHIISDGVSVNILMKDLSRIYEGNEPDPLSIQYKDFAVWQQSDIQKRNIKSQEAYWLDQFHSDI
PVLDMPADYERPAIRDYEGESFEFLIPEHLKQRLSQMEEDTGATLYMILLAAYTILLSRYSGQEDIIVGTPSAGRTHLDVEPVVGMFVNTL
VIRNHPAGRKTFDAYLNEVKENMLNAYKNQDYPLEELIQHLHLPKDSSRNPLFDTMFVLQNLDHAELTEDSLQLKPYSFHHPVAKFDLTLS
IQADQDNYHGLFEYSKKLFKKSRIEVLSNDYLHILSAILEQPSILIEHIGLSGSNEEEENALDSIQLNF

Accordingly, preferably the microorganism comprises a gene encoding the amino acid sequence substantially as set out in SEQ ID No: 10, or a variant or fragment thereof.

The microorganism may comprise a gene coding for a protein sequence with at least 90% sequence identity to SEQ ID NO: 9 or at least 90% sequence identity to SEQ ID NO: 10, such as at least 95% sequence identity to SEQ ID NO: 9, at least 95% sequence identity to SEQ ID NO: 10, at least 98% sequence identity to SEQ ID NO: 9, at least 98% sequence identity to SEQ ID NO: 10, at least 99% sequence identity to SEQ ID NO: 9, at least 99% sequence identity to SEQ ID NO: 10, 100% sequence identity to SEQ ID NO: 9 or 100% sequence identity to SEQ ID NO: 10.

In another embodiment, the microorganism of the invention may comprise 16S rDNA.

Thus, in another preferred embodiment, the microorganism may comprise 16S rDNA of NCIMB 42971, which is provided herein as SEQ ID No: 1, as follows:

[SEQ ID No: 1]
TTTATCGGAGAGTTTGATCCTGGCTCAGGACGAACGCTGGCGGCGTGCCTAATACATGCAAGTCGAGCGGACAGATGGGAGC
TTGCTCCCTGATGTTAGCGGCGGACGGGTGAGTAACACGTGGGTAACCTGCCTGTAAGACTGGGATAACTCCGGGAAACCGG
GGCTAATACCGGATGGTTGTCTGAACCGCATGGTTCAGACATGAAAGGTGGCTTCGGCTACCACTTACAGATGGACCCGCGG
CGCATTAGCTAGTTGGTGAGGTAACGGCTCACCAAGGCGACGATGCGTAGCCGACCTGAGAGGGTGATCGGCCACACTGGGA
CTGAGACACGGCCCAGACTCCTACGGGAGGCAGCAGTAGGGAATCTTCCGCAATGGACGAAAGTCTGACGGAGCAACGCCGC
GTGAGTGATGAAGGTTTTCGGATCGTAAAGCTCTGTTGTTAGGGAAGAACAAGTGCCGTTCAAATAGGGCGGCACCTTGACG
GTACCTAACCAGAAAGCCACGGCTAACTACGTGCCAGCAGCCGCGGTAATACGTAGGTGGCAAGCGTTGTCCGGAATTATTG
GGCGTAAAGGGCTCGCAGGCGGTTTCTTAAGTCTGATGTGAAAGCCCCCGGCTCAACCGGGGAGGGTCATTGGAAACTGGGG
AACTTGAGTGCAGAAGAGGAGAGTGGAATTCCACGTGTAGCGGTGAAATGCGTAGAGATGTGGAGGAACACCAGTGGCGAAG
GCGACTCTCTGGTCTGTAACTGACGCTGAGGAGCGAAAGCGTGGGGAGCGAACAGGATTAGATACCCTGGTAGTCCACGCCG
TAAACGATGAGTGCTAAGTGTTAGGGGGTTTCCGCCCCTTAGTGCTGCAGCTAACGCATTAAGCACTCCGCCTGGGGAGTAC
GGTCGCAAGACTGAAACTCAAAGGAATTGACGGGGGCCCGCACAAGCGGTGGAGCATGTGGTTTAATTCGAAGCAACGCGAA
GAACCTTACCAGGTCTTGACATCCTCTGACAATCCTAGAGATAGGACGTCCCCTTCGGGGGCAGAGTGACAGGTGGTGCATG
GTTGTCGTCAGCTCGTGTCGTGAGATGTTGGGTTAAGTCCCGCAACGAGCGCAACCCTTGATCTTAGTTGCCAGCATTCAGT
TGGGCACTCTAAGGTGACTGCCGGTGACAAACCGGAGGAAGGTGGGGATGACGTCAAATCATCATGCCCCTTATGACCTGGG
CTACACACGTGCTACAATGGACAGAACAAAGGGCAGCGAAACCGCGAGGTTAAGCCAATCCCACAAATCTGTTCTCAGTTCG
GATCGCAGTCTGCAACTCGACTGCGTGAAGCTGGAATCGCTAGTAATCGCGGATCAGCATGCCGCGGTGAATACGTTCCCGG
GCCTTGTACACACCGCCCGTCACACCACGAGAGTTTGTAACACCCGAAGTCGGTGAGGTAACCTTTATGGAGCCAGCCGCCG
AAGGTGGGACAGATGATTGGGGTGAAGTCGTAACAAGGTAGCCGTATCGGAAGGTGCGGCTGGATCACCTCCTTTCTAA

Accordingly, preferably the microorganism comprises the nucleotide sequence substantially as set out in SEQ ID No: 1, or a variant or fragment thereof.

In another preferred embodiment, the microorganism may comprise 16S rDNA of NCIMB 42972, which is provided herein as SEQ ID No: 2, as follows:

[SEQ ID No: 2]
TTTATCGGAGAGTTTGATCCTGGCTCAGGACGAACGCTGGCGGCGTGCCTAATACATGCAAGTCGAGCGGACAGATGGGAGC
TTGCTCCCTGATGTTAGCGGCGGACGGGTGAGTAACACGTGGGTAACCTGCCTGTAAGACTGGGATAACTCCGGGAAACCGG
GGCTAATACCGGATGGTTGTCTGAACCGCATGGTTCAGACATAAAAGGTGGCTTCGGCTACCACTTACAGATGGACCCGCGG
CGCATTAGCTAGTTGGTGAGGTAACGGCTCACCAAGGCGACGATGCGTAGCCGACCTGAGAGGGTGATCGGCCACACTGGGA
CTGAGACACGGCCCAGACTCCTACGGGAGGCAGCAGTAGGGAATCTTCCGCAATGGACGAAAGTCTGACGGAGCAACGCCGC
GTGAGTGATGAAGGTTTTCGGATCGTAAAGCTCTGTTGTTAGGGAAGAACAAGTGCCGTTCAAATAGGGCGGCACCTTGACG
GTACCTAACCAGAAAGCCACGGCTAACTACGTGCCAGCAGCCGCGGTAATACGTAGGTGGCAAGCGTTGTCCGGAATTATTG
GGCGTAAAGGGCTCGCAGGCGGTTTCTTAAGTCTGATGTGAAAGCCCCCGGCTCAACCGGGGAGGGTCATTGGAAACTGGGG
AACTTGAGTGCAGAAGAGGAGAGTGGAATTCCACGTGTAGCGGTGAAATGCGTAGAGATGTGGAGGAACACCAGTGGCGAAG
GCGACTCTCTGGTCTGTAACTGACGCTGAGGAGCGAAAGCGTGGGGAGCGAACAGGATTAGATACCCTGGTAGTCCACGCCG
TAAACGATGAGTGCTAAGTGTTAGGGGGTTTCCGCCCCTTAGTGCTGCAGCTAACGCATTAAGCACTCCGCCTGGGGAGTAC
GGTCGCAAGACTGAAACTCAAAGGAATTGACGGGGGCCCGCACAAGCGGTGGAGCATGTGGTTTAATTCGAAGCAACGCGAA
GAACCTTACCAGGTCTTGACATCCTCTGACAATCCTAGAGATAGGACGTCCCCTTCGGGGGCAGAGTGACAGGTGGTGCATG
GTTGTCGTCAGCTCGTGTCGTGAGATGTTGGGTTAAGTCCCGCAACGAGCGCAACCCTTGATCTTAGTTGCCAGCATTCAGT
TGGGCACTCTAAGGTGACTGCCGGTGACAAACCGGAGGAAGGTGGGGATGACGTCAAATCATCATGCCCCTTATGACCTGGG
CTACACACGTGCTACAATGGACAGAACAAAGGGCAGCGAAACCGCGAGGTTAAGCCAATCCCACAAATCTGTTCTCAGTTCG
GATCGCAGTCTGCAACTCGACTACGTGAAGCTGGAATCGCTAGTAATCGCGGATCAGCATGCCGCGGTGAATACGTTCCCGG
GCCTTGTACACACCGCCCGTCACACCACGAGAGTTTGTAACACCCGAAGTCGGTGAGGTAACCTTTTAGGAGCCAGCCGCCG
AAGGTGGGACAGATGATTGGGGTGAAGTCGTAACAAGGTAGCCGTATCGGAAGGTGCGGCTGGATCACCTCCTTTCTAA

Accordingly, preferably the microorganism comprises the nucleotide sequence substantially as set out in SEQ ID No: 2, or a variant or fragment thereof.

Thus, in another preferred embodiment, the microorganism may comprise 16S rDNA of NCIMB 42973, which is provided herein as SEQ ID No: 3, as follows:

[SEQ ID No: 3]
TTTATCGGAGAGTTTGATCCTGGCTCAGGACGAACGCTGGCGGCGTGCCTAATACATGCAAGTCGAGCGGACAGATGGGAGC
TTGCTCCCTGATGTTAGCGGCGGACGGGTGAGTAACACGTGGGTAACCTGCCTGTAAGACTGGGATAACTCCGGGAAACCGG
GGCTAATACCGGATGGTTGTCTGAACCGCATGGTTCAGACATAAAAGGTGGCTTCGGCTACCACTTACAGATGGACCCGCGG
CGCATTAGCTAGTTGGTGAGGTAACGGCTCACCAAGGCGACGATGCGTAGCCGACCTGAGAGGGTGATCGGCCACACTGGGA
CTGAGACACGGCCCAGACTCCTACGGGAGGCAGCAGTAGGGAATCTTCCGCAATGGACGAAAGTCTGACGGAGCAACGCCGC
GTGAGTGATGAAGGTTTTCGGATCGTAAAGCTCTGTTGTTAGGGAAGAACAAGTGCCGTTCAAATAGGGCGGCACCTTGACG
GTACCTAACCAGAAAGCCACGGCTAACTACGTGCCAGCAGCCGCGGTAATACGTAGGTGGCAAGCGTTGTCCGGAATTATTG
GGCGTAAAGGGCTCGCAGGCGGTTTCTTAAGTCTGATGTGAAAGCCCCCGGCTCAACCGGGGAGGGTCATTGGAAACTGGGG
AACTTGAGTGCAGAAGAGGAGAGTGGAATTCCACGTGTAGCGGTGAAATGCGTAGAGATGTGGAGGAACACCAGTGGCGAAG
GCGACTCTCTGGTCTGTAACTGACGCTGAGGAGCGAAAGCGTGGGGAGCGAACAGGATTAGATACCCTGGTAGTCCACGCCG
TAAACGATGAGTGCTAAGTGTTAGGGGGTTTCCGCCCCTTAGTGCTGCAGCTAACGCATTAAGCACTCCGCCTGGGGAGTAC
GGTCGCAAGACTGAAACTCAAAGGAATTGACGGGGGCCCGCACAAGCGGTGGAGCATGTGGTTTAATTCGAAGCAACGCGAA
GAACCTTACCAGGTCTTGACATCCTCTGACAATCCTAGAGATAGGACGTCCCCTTCGGGGGCAGAGTGACAGGTGGTGCATG
GTTGTCGTCAGCTCGTGTCGTGAGATGTTGGGTTAAGTCCCGCAACGAGCGCAACCCTTGATCTTAGTTGCCAGCATTCAGT
TGGGCACTCTAAGGTGACTGCCGGTGACAAACCGGAGGAAGGTGGGGATGACGTCAAATCATCATGCCCCTTATGACCTGGG
CTACACACGTGCTACAATGGACAGAACAAAGGGCAGCGAAACCGCGAGGTTAAGCCAATCCCACAAATCTGTTCTCAGTTCG
GATCGCAGTCTGCAACTCGACTGCGTGAAGCTGGAATCGCTAGTAATCGCGGATCAGCATGCCGCGGTGAATACGTTCCCGG
GCCTTGTACACACCGCCCGTCACACCACGAGAGTTTGTAACACCCGAAGTCGGTGAGGTAACCTTTATGGAGCCAGCCGCCG
AAGGTGGGACAGATGATTGGGGTGAAGTCGTAACAAGGTAGCCGTATCGGAAGGTGCGGCTGGATCACCTCCTTTCTAA

Accordingly, preferably the microorganism comprises the nucleotide sequence substantially as set out in SEQ ID No: 3, or a variant or fragment thereof.

In another preferred embodiment, the microorganism may comprise 16S rDNA of NCIMB 42974, which is provided herein as SEQ ID No: 4, as follows:

[SEQ ID No: 4]
ACGATGCGTAGCCGACCTGAGAGGGTGATCGGCCACACTGGGACTGAGACACGGCCCAGACTCCTACGGGAGGCAGCAGTAG
GGAATCTTCCGCAATGGACGAAAGTCTGACGGAGCAACGCCGCGTGAGTGATGAAGGTTTTCGGATCGTAAAGCTCTGTTGT
TAGGGAAGAACAAGTACCGTTCGAATAGGGCGGTACCTTGACGGTACCTAACCAGAAAGCCACGGCTAACTACGTGCCAGCA
GCCGCGGTAATACGTAGGTGGCAAGCGTTGTCCGGAATTATTGGGCGTAAAGGGCTCGCAGGCGGTTTCTTAAGTCTGATGT
GAAAGCCCCCGGCTCAACCGGGGAGGGTCATTGGAAACTGGGGAACTTGAGTGCAGAAGAGGAGAGTGGAATTCCACGTGTA
GCGGTGAAATGCGTAGAGATGTGGAGGAACACCAGTGGCGAAGGCGACTCTCTGGTCTGTAACTGACGCTGAGGAGCGAAAG
CGTGGGGAGCGAACAGGATTAGATACCCTGGTAGTCCACGCCGTAAACGATGAGTGCTAAGTGTTAGGGGGTTTCCGCCCCT
TAGTGCTGCAGCTAACGCATTAAGCACTCCGCCTGGGGAGTACGGTCGCAAGACTGAAACTCAAAGGAATTGACGGGGGCCC
GCACAAGCGGTGGAGCATGTGGTTTAATTCGAAGCAACGCGAAGAACCTTACCAGGTCTTGACATCCTCTGACAATCCTAGA
GATAGGACGTCCCCTTCGGGGGCAGAGTGACAGGTGGTGCATGGTTGTCGTCAGCTCGTGTCGTGAGATGTTGGGTTAAGTC
CCGCAACGAGCGCAACCCTTGATCTTAGTTGCCAGCATTCAGTTGGGCACTCTAAGGTGACTGCCGGTGACAAACCGGAGGA
AGGTGGGGATGACGTCAAATCATCATGCCCCTTATGACCTGGGCTACACACGTGCTACAATGGACAGAACAAAGGGCAGCGA
AACCGCGAGGTTAAGCCAATCCCACAAATCTGTTCTCAGTTCGGATCGCAGTCTGCAACTCGACTGCGTGAAGCTGGAATCG
CTAGTAATCGCGGATCAGCATGCCGCGGTGAATACGTTCCCGGGCCTTGTACACACCGCCCGTCACACCACGAGAGTTTGTA
ACACCCGAAGTCGGTGAGGTAACCTTTTAGGAGCCAGCCGCCGAAGGTGGGACAGATGATTGGGGTGAAGTCGTAACAAGGT
AGCCGTATCGGAAGGTGCGGCTGGATCACCTCCTTTCTAAGGA

Accordingly, preferably the microorganism comprises the nucleotide sequence substantially as set out in SEQ ID No: 4, or a variant or fragment thereof.

In another preferred embodiment, the microorganism may comprise 16S rDNA of NCIMB 43393, which is provided herein as SEQ ID No: 5, as follows:

[SEQ ID No: 5]
TTTATCGGAGAGTTTGATCCTGGCTCAGGACGAACGCTGGCGGCGTGCCTAATACATGCAAGTCGAGCGGACAGATGGGAGC
TTGCTCCCTGATGTTAGCGGCGGACGGGTGAGTAACACGTGGGTAACCTGCCTGTAAGACTGGGATAACTCCGGGAAACCGG
GGCTAATACCGGATGGTTGTCTGAACCGCATGGTTCAGACATAAAAGGTGGCTTCGGCTACCACTTACAGATGGACCCGCGG
CGCATTAGCTAGTTGGTGAGGTAACGGCTCACCAAGGCGACGATGCGTAGCCGACCTGAGAGGGTGATCGGCCACACTGGGA
CTGAGACACGGCCCAGACTCCTACGGGAGGCAGCAGTAGGGAATCTTCCGCAATGGACGAAAGTCTGACGGAGCAACGCCGC
GTGAGTGATGAAGGTTTTCGGATCGTAAAGCTCTGTTGTTAGGGAAGAACAAGTGCCGTTCAAATAGGGCGGCACCTTGACG
GTACCTAACCAGAAAGCCACGGCTAACTACGTGCCAGCAGCCGCGGTAATACGTAGGTGGCAAGCGTTGTCCGGAATTATTG
GGCGTAAAGGGCTCGCAGGCGGTTTCTTAAGTCTGATGTGAAAGCCCCCGGCTCAACCGGGGAGGGTCATTGGAAACTGGGG
AACTTGAGTGCAGAAGAGGAGAGTGGAATTCCACGTGTAGCGGTGAAATGCGTAGAGATGTGGAGGAACACCAGTGGCGAAG
GCGACTCTCTGGTCTGTAACTGACGCTGAGGAGCGAAAGCGTGGGGAGCGAACAGGATTAGATACCCTGGTAGTCCACGCCG
TAAACGATGAGTGCTAAGTGTTAGGGGGTTTCCGCCCCTTAGTGCTGCAGCTAACGCATTAAGCACTCCGCCTGGGGAGTAC
GGTCGCAAGACTGAAACTCAAAGGAATTGACGGGGGCCCGCACAAGCGGTGGAGCATGTGGTTTAATTCGAAGCAACGCGAA
GAACCTTACCAGGTCTTGACATCCTCTGACAATCCTAGAGATAGGACGTCCCCTTCGGGGGCAGAGTGACAGGTGGTGCATG
GTTGTCGTCAGCTCGTGTCGTGAGATGTTGGGTTAAGTCCCGCAACGAGCGCAACCCTTGATCTTAGTTGCCAGCATTCAGT
TGGGCACTCTAAGGTGACTGCCGGTGACAAACCGGAGGAAGGTGGGGATGACGTCAAATCATCATGCCCCTTATGACCTGGG
CTACACACGTGCTACAATGGACAGAACAAAGGGCAGCGAAACCGCGAGGTTAAGCCAATCCCACAAATCTGTTCTCAGTTCG
GATCGCAGTCTGCAACTCGACTGCGTGAAGCTGGAATCGCTAGTAATCGCGGATCAGCATGCCGCGGTGAATACGTTCCCGG
GCCTTGTACACACCGCCCGTCACACCACGAGAGTTTGTAACACCCGAAGTCGGTGAGGTAACCTTTATGGAGCCAGCCGCCG
AAGGTGGGACAGATGATTGGGGTGAAGTCGTAACAAGGTAGCCGTATCGGAAGGTGCGGCTGGATCACCTCCTTTCTAA

Accordingly, preferably the microorganism comprises the nucleotide sequence substantially as set out in SEQ ID No: 5, or a variant or fragment thereof.

In another preferred embodiment, the microorganism may comprise 16S rDNA of NCIMB 43392, which is provided herein as SEQ ID No: 6, as follows:

[SEQ ID No: 6]
TTTATCGGAGAGTTTGATCCTGGCTCAGGACGAACGCTGGCGGCGTGCCTAATACATGCAAGTCGAGCGGACAGATGGGAGC
TTGCTCCCTGATGTTAGCGGCGGACGGGTGAGTAACACGTGGGTAACCTGCCTGTAAGACTGGGATAACTCCGGGAAACCGG
GGCTAATACCGGATGGTTGTCTGAACCGCATGGTTCAGACATAAAAGGTGGCTTCGGCTACCACTTACAGATGGACCCGCGG
CGCATTAGCTAGTTGGTGAGGTAACGGCTCACCAAGGCGACGATGCGTAGCCGACCTGAGAGGGTGATCGGCCACACTGGGA
CTGAGACACGGCCCAGACTCCTACGGGAGGCAGCAGTAGGGAATCTTCCGCAATGGACGAAAGTCTGACGGAGCAACGCCGC
GTGAGTGATGAAGGTTTTCGGATCGTAAAGCTCTGTTGTTAGGGAAGAACAAGTGCCGTTCAAATAGGGCGGCACCTTGACG
GTACCTAACCAGAAAGCCACGGCTAACTACGTGCCAGCAGCCGCGGTAATACGTAGGTGGCAAGCGTTGTCCGGAATTATTG
GGCGTAAAGGGCTCGCAGGCGGTTTCTTAAGTCTGATGTGAAAGCCCCCGGCTCAACCGGGGAGGGTCATTGGAAACTGGGG
AACTTGAGTGCAGAAGAGGAGAGTGGAATTCCACGTGTAGCGGTGAAATGCGTAGAGATGTGGAGGAACACCAGTGGCGAAG
GCGACTCTCTGGTCTGTAACTGACGCTGAGGAGCGAAAGCGTGGGGAGCGAACAGGATTAGATACCCTGGTAGTCCACGCCG
TAAACGATGAGTGCTAAGTGTTAGGGGGTTTCCGCCCCTTAGTGCTGCAGCTAACGCATTAAGCACTCCGCCTGGGGAGTAC
GGTCGCAAGACTGAAACTCAAAGGAATTGACGGGGGCCCGCACAAGCGGTGGAGCATGTGGTTTAATTCGAAGCAACGCGAA
GAACCTTACCAGGTCTTGACATCCTCTGACAATCCTAGAGATAGGACGTCCCCTTCGGGGGCAGAGTGACAGGTGGTGCATG
GTTGTCGTCAGCTCGTGTCGTGAGATGTTGGGTTAAGTCCCGCAACGAGCGCAACCCTTGATCTTAGTTGCCAGCATTCAGT
TGGGCACTCTAAGGTGACTGCCGGTGACAAACCGGAGGAAGGTGGGGATGACGTCAAATCATCATGCCCCTTATGACCTGGG
CTACACACGTGCTACAATGGACAGAACAAAGGGCAGCGAAACCGCGAGGTTAAGCCAATCCCACAAATCTGTTCTCAGTTCG
GATCGCAGTCTGCAACTCGACTGCGTGAAGCTGGAATCGCTAGTAATCGCGGATCAGCATGCCGCGGTGAATACGTTCCCGG
GCCTTGTACACACCGCCCGTCACACCACGAGAGTTTGTAACACCCGAAGTCGGTGAGGTAACCTTTATGGAGCCAGCCGCCG
AAGGTGGGACAGATGATTGGGGTGAAGTCGTAACAAGGTAGCCGTATCGGAAGGTGCGGCTGGATCACCTCCTTTCTAA

Accordingly, preferably the microorganism comprises the nucleotide sequence substantially as set out in SEQ ID No: 6, or a variant or fragment thereof.

The microorganism may comprise 16S rDNA that is more than 98% sequence identity, such as more than 98% sequence identity or 100% sequence identity, to SEQ ID NO: 1 and/or more than 98% sequence identity such as more than 98% sequence identity or 100% sequence identity, to SEQ ID NO: 2 and/or more than 98% sequence identity such as more than 98% sequence identity or 100% sequence identity, to SEQ ID NO: 3 and/or more than 98% sequence identity such as more than 98% sequence identity or 100% sequence identity, to SEQ ID NO: 4 and/or more than 98% sequence identity such as more than 98% sequence identity or 100% sequence identity, to SEQ ID NO: 5 and/or more than 98% sequence identity such as more than 98% sequence identity or 100% sequence identity, to SEQ ID NO: 6.

In one embodiment, the microorganism may comprise one or more of the nucleotide sequences selected from the group consisting of: SEQ ID No: 1 to 6, or variants or fragments thereof.

In one embodiment, the microorganism may comprise two or more of the nucleotide sequences selected from the group consisting of: SEQ ID No: 1 to 6, or variants or fragments thereof.

In one embodiment, the microorganism may comprise three or more of the nucleotide sequences selected from the group consisting of: SEQ ID No: 1 to 6, or variants or fragments thereof.

In one embodiment, the microorganism may comprise four or more of the nucleotide sequences selected from the group consisting of: SEQ ID No: 1 to 6, or variants or fragments thereof.

In one embodiment, the microorganism may comprise five or more of the nucleotide sequences selected from the group consisting of: SEQ ID No: 1 to 6, or variants or fragments thereof.

In one embodiment, the microorganism may comprise the nucleotide sequences selected from the group consisting of: SEQ ID No: 1 to 6, or variants or fragments thereof.

Strains

The B. amyloliquefaciens strain that is used is selected from a group consisting of: SG57, SG137, SG185, SG277 and SG297. Most preferably, the B. amyloliquefaciens strain is SG277 or SG297. The B. subtilis strain is SG140.

In one embodiment, one or more strains of B. amyloliquefaciens, or extracellular material produced by the cell or disrupted cell homogenate, is used. In other words, any B. amyloliquefaciens strain selected from a group consisting of: SG57, SG137, SG185, SG277 and SG297, may be used. Alternatively, in another embodiment, more than one B. amyloliquefaciens strain selected from a group consisting of: SG57, SG137, SG185, SG277 and SG297, may be used. For example, SG277 and SG297 could be used simultaneously, or SG137 and SG57 could be used simultaneously, and so on.

In yet another embodiment, one or more strains of B. amyloliquefaciens may be used in combination with B. subtilis, or extracellular material produced by the corresponding cell or disrupted cell homogenate therefrom. For example, B. amyloliquefaciens strain SG277 may be used with B. subtilis strain SG140.

The most preferred strains are strains deposited under the Budapest treaty at the NCIMB, Ferguson Building, Craibstone Estate, Bucksburn, Aberdeen, AB21 9YA on 15 Feb. 2018 and 10 May 2019.

Designation number: NCIMB 42971—Referred to herein as: B. amyloliquefaciens SG277.

Designation number: NCIMB 42972—Referred to herein as: B. amyloliquefaciens SG297.

Designation number: NCIMB 42973—Referred to herein as: B. amyloliquefaciens SG185.

Designation number: NCIMB 42974—Referred to herein as: B. subtilis SG140.

Designation number: NCIMB 43392—Referred to herein as B. amyloliquefaciens SG57.

Designation number: NCIMB 43393—Referred to herein as B. amyloliquefaciens SG137.

Recent changes in the bacterial taxonomy include the reclassification of Bacillus amyloliquefaciens strains as Bacillus velezensis. The present application discloses stains designated SG57, SG137, SG185, SG277 and SG297, and these strains have been designated Bacillus amyloliquefaciens without taking recent changes in the taxonomy into account. It should therefore be understood that the species designation Bacillus amyloliquefaciens as used in the present description and claims includes strains that a taxonomy expert would designate as Bacillus velezensis strains.

In yet another embodiment, one or more strains of B. amyloliquefaciens may be used in combination with one or more strains of B. subtilis, or extracellular material produced by the corresponding cell or disrupted cell homogenate therefrom. For example in compositions of the invention comprising two strains, B. amyloliquefaciens strain NCIMB 42971 may be used with B. amyloliquefaciens NCIMB 42972, B. amyloliquefaciens NCIMB 42973, B. subtilis NCIMB 42974,

B. amyloliquefaciens NCIMB 43392 or B. amyloliquefaciens NCIMB 43393; B. amyloliquefaciens strain NCIMB 42972 may be used with B. amyloliquefaciens NCIMB 42973, B. subtilis NCIMB 42974, B. amyloliquefaciens NCIMB 43392 or B. amyloliquefaciens NCIMB 43393; B. amyloliquefaciens strain NCIMB 42973 may be used with B. subtilis NCIMB 42974, B. amyloliquefaciens NCIMB 43392 or B. amyloliquefaciens NCIMB 43393; B. subtilis NCIMB 42974 may be used with B. amyloliquefaciens NCIMB 43392 or B. amyloliquefaciens NCIMB 43393; or B. amyloliquefaciens NCIMB 43392 may be used with B. amyloliquefaciens NCIMB 43393.

The composition may comprise a Bacillus amyloliquefaciens strain, and the Bacillus amyloliquefaciens strain is selected from the strains deposited as NCIMB 42971, NCIMB 42972, NCIMB 42973, NCIMB 43392 or NCIMB 43393. The composition may comprise a Bacillus subtilis strain, and the Bacillus subtilis strain is the strain deposited as NCIMB 42974. All of the one or more microorganisms may be selected from the Bacillus amyloliquefaciens strains deposited as NCIMB 42971, NCIMB 42972, NCIMB 42973, NCIMB 43392 or NCIMB 43393; and the Bacillus subtilis strain deposited as NCIMB 42974.

Compositions

The compositions of the invention comprise one or more of the B. amyloliquefaciens strains selected from a group consisting of: NCIMB 42971, NCIMB 42972 NCIMB 42973, NCIMB 43392, NCIMB 43393 and/or the B. subtilis strain NCIMB 42974, and/or extracellular material produced by the live cells, and further one or more food grade ingredients.

A composition according to the invention may for instance be characterized by that it contains 10² to 10¹⁵, preferably 10⁶ or 10⁸ to 10¹², in particular 10⁸ to 10¹⁰, Bacillus cells (either in the form of vegetative cells or spores or a mixture thereof). Reference value is a unit of administration, for instance a tablet, a capsule or a sachet. Preferably, the composition is prepared for oral administration. The Bacillus cells are suitably lyophilized or spray dried.

The preparation of a composition according to the invention can be made in a way being usual in these technologies. Suitable solid or liquid preparation forms are for instance granulates, powders, dragees, tablets, (micro) capsules, suppositories, syrups, juices, suspensions or emulsions, for the production of which usual means are used, such as carrier substances, binding, coating, swelling, sliding or lubricating agents, tasting agents, sweeteners and solution mediators. As auxiliary substances are named here sodium aluminosilicate, magnesium carbonate, titanium dioxide, lactose, mannite and other sugars, talcum, milk protein, gelatin, starch, cellulose and derivatives, animal and vegetable oils such as cod-liver oil, sunflower oil, peanut oil or sesame oil, polyethylene glycols and solvents, such as sterile water and mono- or multi-valent alcohols, for instance glycerine. A composition according to the invention can be produced such that cells of at least one Bacillus strain used according to the invention is mixed in a defined dose with a pharmaceutically suitable and physiologically well tolerated carrier and possibly further suitable active, additional or auxiliary substances, and is prepared in the desired form of administration. Carriers are in particular substances, which are selected from the group comprising “maltodextrin, microcrystalline cellulose, starch, in particular corn starch, levulose, lactose, dextrose, and mixtures of such substances”. The composition may contain 0.1 to 95 per-cent by weight carrier and 5 to 99.9 percent by weight Bacillus cells (either in the form of vegetative cells or spores or a mixture thereof), relative to the total amount of cells and carriers, or consist thereof.

In the case of the food composition, it may be provided that the composition contains 10² to 10¹⁵, preferably 10⁶ to 10⁹, in particular 10⁷ to 10⁹, Bacillus cells (either in the form of vegetative cells or spores or a mixture thereof). Reference value is a unit of administration, for instance a packing unit of a food material to be sold to an end user. The physiologically tolerated carrier will normally be a food material, which in particular is selected from the group comprising “milk products, fermented milk products, milk, yogurt, cheese, cereals, muesli bars, and children's food preparations”.

The invention further concerns a method for the production of a pharmaceutical and/or dietetic composition according to the invention, wherein the lyophilized or not lyophilized, preferably viable Bacillus cells or spores are mixed with the physiologically tolerated carrier and prepared for oral administration.

The composition may further comprise further ingredients known from the art of preparing foods and food supplements, e.g. selected among fillers, nutrients, minerals (especially calcium may by advantageously administered to diarrheal patients); preservatives, stabilizing agents, flavouring and colouring agents.

The composition may be a food supplement, or in the form of a food or food composition.

When the preparation of the invention is in the form of a food supplement, it can be in a form for separate administration, such as a capsule, a tablet, a powder or a similar form, containing preferably a unit dose of the micro-organisms, containing 10²-10¹⁵ cells/dose, preferably 10⁸-10¹¹ cells/dose (where cells are either in the form of vegetative cells or spores or a mixture thereof).

The food supplement can also be in the form of a powder or a similar form, which is added to, or mixed with, a suitable food (composition) or a suitable liquid or solid carrier, for the preparation of a food which is ready for consumption.

For instance, the food supplement can be in the form of a dried powder, which is reconstituted using a suitable liquid, such as water, oral rehydration solution, milk, fruit juice, or similar drink-able liquids. It can also be in the form of a powder which is mixed with solid foods, or foods with a high water-content, such as fermented milk products, for example yoghurt.

The composition of the invention can also be in the form of a food which is ready for consumption. Such a food can for instance be prepared by adding a supplement of the invention as described above to a food or food base known per se; adding the micro-organisms (separately or as a mixture) in the amounts required for administration to a food or food base known per se; or by cultivating the required bacteria in a food medium until a food containing the amount of bacteria required for administration is obtained. The food medium is preferably such that it already forms part of the food, or will form part of the food after fermentation.

In this respect, food or food base can be either fermented or non-fermented.

The composition of the invention can be foods for oral consumption, for instance a total food or an infant formula.

The composition can further contain prebiotic compounds, in particular fibres that lead to the production butyrate/butyric acid, propionate/propionic acid or acetate/acetic acid upon fermentation; nitrogen donors such as proteins; and specific vitamins, minerals and/or trace elements. With respect to the latter, and as can be seen from the examples, the presence of increased or moderately high amounts of vitamin A, K, B₁₂, biotin, Mg, Ca and Zn can be advantageous, as can the presence of folic acid in preparations intended for the treatment of chronic diarrhoea.

The food supplement may further comprise fibres e.g. an amount of at least 0.5 g fibre per 100 g of the total preparation.

As the fibres, the preparation preferably contains a resistant starch or another butyrate generator, as well as a suitable propionate generator such as gums or soy polysaccharides, in the amounts indicated above. Short-chain fatty acids such as butyric acid and propionic acid can also be used as such, preferably in a suitably encapsulated form, or as a physiological equivalent thereof, such as sodium propionate, in an amount of at least 0.1 g per 100 g of the total composition.

Nitrogen, vitamins, minerals and trace elements may also be included e.g. in form of yeast extract.

The composition of the invention can further contain one or more substances that inhibit bacterial adhesion to the epithelial wall of the gastrointestinal tract. Preferably, these compounds are selected from lectins, glycoproteins, mannans, glucans, chitosan and/or derivatives thereof, charged proteins, charged carbohydrates, sialylated compounds and/or adhesion-inhibiting immunoglobulins, galacto-oligosaccharides, as well as modified carbohydrates and modified chi-tin, the latter in amounts of 1-10% w/v, preferably 2-5% w/v of the composition.

Preferred adherence-inhibiting substances are chitosan, carob flour, as well as extracts which are rich in condensed tannin and tannin-derivatives, such as cranberry extract; the amount of tannin in the final product preferably being 10-600 μg/ml.

The composition, in particular if the composition is in the form of a total food, may also contain peptides and/or proteins, in particular proteins that are rich in glutamate and glutamine, lipids, carbohydrates, vitamins, minerals and trace elements. The use of glutamine/glutamate precursors, in amounts corresponding to 0.6-3 g glutamine/100 g product, as well as of small polypeptides that have a high content of glutamines, is preferred. Alternatively, proteins that are rich in glutamine, such as milk proteins, wheat proteins or hydrolysates thereof, can be added.

In one preferred embodiment the composition further comprise glucosamine. Glucosamine is typically included in amounts corresponding to a daily intake in the range of 10-2000 mg, e.g., in the range of 100-2000 mg, e.g., in the range of 250-1500 mg e.g., in the range of 500-1000 mg. In particular preferred embodiments the compositions of the invention is formulated in unit dosage forms, where 1-5 unit dosage forms correspond to a daily dosage of 10² to 10¹⁵, preferably 10⁶ to 10¹², in particular 10⁸ to 10¹¹, Bacillus cells and glucosamine in amounts in the range of 10-2000 mg, e.g., in the range of 100-2000 mg, e.g. in the range of 250-1500 mg e.g., in the range of 500-1000 mg. The glucosamine may be glucosamine hydrochloride or glucosamine phosphate. The skilled person would understand that glucosamine may also be referred to as chitosamine.

The compositions of the invention may be lactose-free. In some embodiments the compositions have a high osmolality (preferably less than 400 mosm/1, more preferably less than 300 mosm/1), in other embodiments the compositions have a lower osmolality e.g. as the compositions disclosed in https://www.ncbi.nlm.nih.gov/pmc/articles/PMC1717650/. In some embodiments the compositions of the invention are kosher, vegetarian or vegan.

In some embodiments the composition of the invention is a pharmaceutical composition comprising a strain of the invention and one or more pharmaceutically acceptable excipients.

The inventors have prepared novel foods, food ingredients, dietary supplements, dietary supplement ingredients, medical foods, foods for special medical purposes, foods for specified health use, foods for special dietary use, health foods, Complementary Medicines Natural Health Products, Natural Health formulations, Natural Health ingredients, and pharmaceutical products, pharmaceutical preparations, pharmaceutical formulations, and pharmaceutical ingredients comprising one of the various B. amyloliquefaciens and B. subtilis strains or any combination of such strains.

Accordingly, there is provided a composition comprising a live or dead spore, or a live or dead vegetative cell, or mixture thereof, of one or more B. amyloliquefaciens strains and/or one or more B. subtilis strains, or extracellular material produced by the live cell, or disrupted cell homogenate; and, one or more food grade ingredients preferably selected among: carrier or vehicle, fillers, stabilizers, nutrients, flavourings, colourings.

The carriers or vehicles are selected among any food grade materials that are inert under the conditions applied during storing and use. Examples of carriers or vehicles includes minerals, such as CaCO₃, NaCl, KCl, CaHPO₄; polymers such as natural or modified starch, pectin, cellulose; sugar such as lactose, sucrose or glucose; flour and skimmed milk powder.

The fillers are selected among ingredients that are inert under the conditions applied. Fillers are typically added to the compositions of the invention to secure that the composition obtain the desired volume.

The stabilizers are selected among food grade ingredients having the ability to stabilize and/or protect the one or more B. amyloliquefaciens strains and/or one or more B. subtilis strains, during production and/or storage. Examples of suitable stabilizers include ascorbic acid and vitamin E.

The nutrients can in principle be selected among any nutrients, provided that the composition does not support growth of the one or more B. amyloliquefaciens strains and/or one or more B. subtilis strains. Typically this means that the composition is dry or at least that the water activity is so low that microbial growth is prevented. Example of nutrients includes minerals, vitamins, sugars, proteins, milk or fractions thereof including milk powders, flour, honey and juice.

The flavourings and colorants are selected among food grade flavourings and colorants as known in the area.

The composition may be a food, food ingredient, dietary supplement, dietary supplement ingredient, medical food, food for special medical purposes, food for specified health use, food for special dietary use, health food, Complementary Medicine; Natural Health Product, Natural Health formulation, Natural Health ingredient, pharmaceutical product, pharmaceutical preparation, pharmaceutical formulation, or a pharmaceutical ingredient.

The composition may be a probiotic composition comprising or consisting of live or dead cells, or spores, or compounds originating from the cells or spores. The composition may comprise one or more food grade ingredients selected from fillers and stabilizing agents. The composition may be provided in a unit dosage formulation, such as a capsule, tablet or sachet. Each unit dosage formulation may comprise 10⁸ to 10¹⁰ CFU of the one or more microorganisms. The composition may be a food composition, comprising at least one nutrient and/or vitamin in addition to the one or more microbial strains. The food composition may comprise a CFU count corresponding to 10⁸ to 10¹⁰ CFU per serving.

The one or more microorganisms may be provided in lyophilized or spray-dried form. The composition may further comprise glucosamine. The composition may comprise glucosamine corresponding to a daily dosage of 10-2000 mg, optionally in the range of 100-2000 mg, or in the range of 250-1500 mg, or in the range of 500-1000 mg. The glucosamine may be glucosamine hydrochloride or glucosamine phosphate.

Preparation

The one or more Bacillus strains used in the compositions of the invention may be obtained in any manner known per se, such as cultivating them in suitable media, preferably in such a way and using such media that the microbial preparations thus obtained are suitable for administration to humans and/or animals.

Cultivation of the one or more Bacillus strains used in the compositions of the invention may be performed in standard fermentation equipment suitable for fermenting Bacilli, as will be known in the art.

After cultivation, the one or more Bacillus strains used in the compositions of the invention are recovered from the fermentation broth and turned into a composition of the invention using techniques known in the art, or alternatively the complete fermentation broth may be turned into the composition according to the invention.

The one or more Bacillus strains may be freeze-dried. When microorganisms are freeze-dried it is important that methods that secure a satisfactory high viable cell number and anti-pathogenic activity is used. Such techniques are known in the art and such methods known in the art are also applicable in the present invention.

Freeze dried preparations may contain suitable adjuvants known per se, for example cryoprotectants such as nutriose, maltose or prebiotics (e.g. galactooligosaccharides).

The one or more Bacillus strains used in the compositions of the invention may be spray dried meaning that the Bacillus cells are dried using a spray drying or atomizing method (synonymous), wherein a suspension of one or more Bacillus strains used in the compositions of the invention is dispersed into fine mist-like droplets, for example, and a powder can be obtained.

During spray drying according to the invention, a solution or suspension containing one or more Bacillus strains used in the compositions of the invention cells is sprayed into a hot drying medium, whereby it is dried. The mixture to be sprayed can be present in the form of a solution, an emulsion, a suspension or dispersion. It is atomized into millions of individual droplets with the aid of a nozzle or a spraying wheel, drastically increasing the surface. The solvent, such as water, is immediately evaporated by the hot air and is discharged. Moreover, the one or more Bacillus strains used in the compositions of the invention may be spray-dried alone.

The spray drying or atomization method can be distinguished from other drying methods since the use of a nozzle or similarly acting means is required, such as a unary nozzle, hollow cone nozzle, pressure nozzle, binary nozzle externally mixing, pneumatic nozzle, binary nozzle internally mixing, atomizing disk or ultrasonic atomizer.

Spray drying methods are described in the prior art and are familiar to the person skilled in the art (see Gardiner et al., Teixeira et al. (supra) or EP74050 and EP285682). Devices are known and described as relevant, such as the mini spray dryer B-191 or B-290 by Buechi Labortechnik AG (Germany) or SD-6.3-R by GEA Niro (Denmark). It is further known that arbitrary adjuvants and additives can be used. WO2012/168468 describes preferred methods for spray drying bacterial such as the one or more Bacillus strains used in the compositions of the invention.

Dosage and Administration

The compositions according to the invention are administered to individuals at risk of obtaining an infection with Clostridioides difficile or at risk of obtaining asymptomatic colonization of C. difficile.

Individuals at risk of obtaining an infection with Clostridioides difficile include healthy individuals such as young people, elderly people or individuals having a reduced immune defense e.g., as a result of other diseases, medical treatment or because they have been exposed to severe conditions e.g., stress, fatigue or malnutrition. The compositions according to the invention may be administered to this group of individuals in order to prevent an infection with Clostridioides difficile, or reduce the duration or severity of an infection with C. difficile, or prevent or reduce asymptomatic C. difficile colonization.

The compositions of the invention are administered to the above-mentioned individuals in amounts so that the individuals receive a daily dose of 10² to 10¹⁵, preferably 10⁶ or 10⁸ to 10¹² in particular 10⁸ to 10¹⁰, Bacillus cells.

In one preferred embodiment, the compositions are formulated as discrete dosage forms e.g. as tablets, capsules or sachets. Each dosage form is formulated so it comprises 10⁸ to 10¹⁰, Bacillus cells. It is preferred that 1-2 of such discrete dosage forms are administered daily to each individual.

In another preferred embodiment, the compositions are formulated as food compositions, which composition is formulated so that one serving comprises 10⁸ to 10¹⁰, Bacillus cells, and that each individual receiving one serving per day.

Administered in this way the composition of the invention can prevent C. difficile infection, or reduce the duration and severity of C. difficile infection, or prevent or reduce asymptomatic colonization of C. difficile.

In an aspect, there is provided a use of the composition described herein for the prevention of C. difficile infection in an individual at risk of obtaining such an infection, or reduction of duration or severity of a C. difficile infection, or prevention or reduction of asymptomatic C. difficile colonization.

10⁸ to 10¹⁰ CFU Bacillus cells may be administered daily. The composition may prevent or reduce C. difficile infection in the gastrointestinal tract of a subject, preferably a human subject.

In another aspect, there is provided the composition described herein for use in the prevention of C. difficile infection in an individual at risk of obtaining such an infection, or reduction of duration or severity of a C. difficile infection, or prevention or reduction of asymptomatic C. difficile colonization.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1-27 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45° C. followed by at least one wash in 0.2× SSC/0.1% SDS at approximately 20-65° C. Alternatively, a substantially similar polypeptide may differ by at least 1, but less than 5, 10, 20, 50 or 100 amino acids from the sequences shown in, for example, any of SEQ ID Nos: 1-27 that correspond to amino acid sequences.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

### EXAMPLES

### Example 1

To evaluate the ability of B. amyloliquefaciens (SG277) to suppress or inhibit C. difficile infection (CDI) in vivo a mouse trial was conducted.

Mouse C57 BL/6 (females, age 10-11 weeks) Source: Charles River Housing: Animals were housed in groups (n=5) until the start of the study. After which they were housed individually in IVCs (independently ventilated cages). Food: Irradiated food pellets and sterile drinking water.

Test Products (TPs):

| | |
|---|---|
| TP1 | SG277 5 × 10⁹ CFU/dose |
| | SG277 grown 16 h at 37° C. in 25 ml BHIB (>90% spores) |
| TP2 | SG277 5 × 10⁹ CFU + germinant (10 mM glucose + L-alanine) |
| TP3 | SG277 2 × 10⁸ CFU (<4% spores) |

Dose: 0.2 ml of the TP by intra-gastric (i.g.) gavage.

TP1 and TP2 were prepared by growth of SG277 (16 h, 37° C.) in 250 ml Bellco flasks with 25 ml BHIB. Using these conditions >90% of culture=spores. The cells were pelleted and suspended in 0.5 ml of supernatant. TP3 was SG277 grown in a test tube in a roller drum (16 h, 37° C.) and under these conditions produced low levels of spore formation. 1 ml of culture was pelleted and suspended in 0.2 ml of supernatant.

Groups: n=5

| | |
|---|---|
| A | TP1 |
| B | TP2 |
| C | TP3 |
| D | placebo |

Analysis: Toxin A in caecum at 48 h

CFU of C. difficile spores in caecum at 48 h

Protocol:

The animals were treated with 30 mg/kg Clindamycin 24 hours before C. difficile challenge. 4 and 1 hours before C. difficile challenge and 1, 7, 13, 25, 31 and 37 hours after the C. difficile challenge the animals were treated with Test products. At 0 hours the animals were give a C. difficile challenge consisting of 100 CFU C. difficile strain CD 630. After 48 hours the Toxin A concentration and CFU C. difficile in caecum of the animals were determined

Results

TABLE 1
Toxin A analysis

| | | Toxin A | Gp average |
|---|---|---|---|
| Group | Mouse | (ng/g caecum) | (ng/g caecum) |
| A | 1 | 216 | 446 |
| TP1 | 2 | 613 | |
| | 3 | 795 | |
| | 4 | 292 | |
| | 5 | 314 | |
| B | 1 | 524 | 141 |
| TP2 | 2 | 0 | |
| | 3 | 123 | |
| | 4 | 56 | |
| | 5 | 0 | |
| C | 1 | 125 | |
| TP3 | 2 | 575 | |
| | 3 | 591 | |
| | 4 | 220 | |
| | 5 | 72 | |
| D | 1 | 1190 | |
| Placebo | 2 | 882 | |
| | 3 | 3112 | |
| | 4 | 935 | |
| | 5 | 2691 | |

TABLE 2
C. difficile CFU

| | | | Gp average |
|---|---|---|---|
| Group | Mouse | CFU/g caecum | (CFU/g) |
| A | 1 | 0 | 0 |
| TP1 | 2 | 0 | |
| | 3 | 0 | |
| | 4 | 0 | |
| | 5 | 0 | |
| B | 1 | 0 | 0 |
| TP2 | 2 | 0 | |
| | 3 | 0 | |
| | 4 | 0 | |
| | 5 | 0 | |
| C | 1 | 2.0 × 10⁴ | 4 × 10³ |
| TP3 | 2 | 0 | |
| | 3 | 0 | |
| | 4 | 0 | |
| | 5 | 0 | |
| D | 1 | 6.5 × 10⁶ | 4.3 × 10⁶ |
| Placebo | 2 | 5.0 × 10⁵ | |
| | 3 | 6.5 × 10⁶ | |
| | 4 | 2.0 × 10⁶ | |
| | 5 | 6.0 × 10⁶ | |

Summary

This experiment showed that SG277 could successfully prevent colonization of C. difficile using caecum CFU and toxins for defining colonization.

### Example 2

This mouse trial was conducted to evaluate the ability of B. amyloliquefaciens (Ba) strains (SG57, SG137, SG277) to suppress or inhibit C. difficile infection (CDI) in vivo.

Species: Mouse C57 BL/6 (females, age 12-13 weeks) Source: Charles River. Housing: Animals were housed in groups (n=5) until the start of the study. After which they were housed individually in IVCs (independently ventilated cages). Food: Irradiated food pellets and sterile drinking water.

Test Products (TPs):
B. amyloliquefaciens SG57, SG137, SG277 in three forms:
i) SP+VC in SUP

Cells of each of the 3 strains was grown for 16 h at 37° C. in 25 ml BHIB The cells were harvested from 50 ml culture and the pellet resuspended in 0.5 ml of supernatant.

I dose=0.1 ml and ˜5×10⁹ CFU consisting of spores (˜70-90%)
ii) SP+VC in PBS

As above but the pellet was suspended in PBS

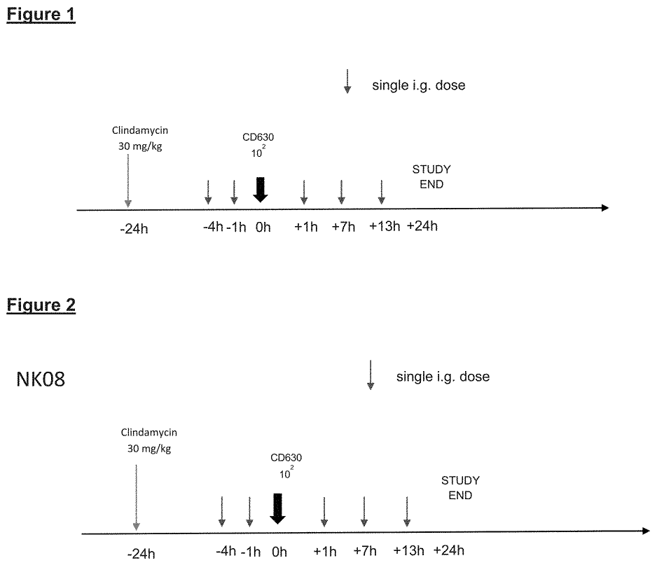

I dose=0.1 ml and ˜5×10⁹ CFU consisting of spores (˜70-90%)
iii) SUP
0.1 ml of supernatant only
C. difficile challenge, administration of the test composition and sampling were made as indicated in FIG. 1.

Challenge: C. difficile strain CD630 (Wüst et al. 1982 J. Clin Microbiol (16) 1096-1101)

Groups: n=4

| | |
|---|---|
| Gp A | SG277 SP + VC in SUP |
| Gp B | SG277 SP + VC in PBS |
| Gp C | SG277 SUP |
| Gp D | SG57 SP + VC in SUP |
| Gp E | SG57 SP + VC in PBS |
| Gp F | SG57 SUP |
| Gp G | SG137 SP + VC in SUP |
| Gp H | SG137 SP + VC in PBS |
| Gp I | SG137 SUP |
| Gp J | PBS (0.1 ml) |

Analysis: CFU of C. difficile spores in caecum at 24 h

Results

TABLE 3
CFU in caecum

| | | CFU/g | CFU/g |
|---|---|---|---|
| Group | Mice | (EtOH Res) | (h^{R}) |
| A | 1 | 0 | 0 |
| | 2 | 0 | 0 |
| | 3 | 0 | 0 |
| | 4 | 0 | 0 |
| B | 1 | 0 | 0 |
| | 2 | 0 | 0 |
| | 3 | 0 | 0 |
| | 4 | 0 | 0 |
| C | 1 | 0 | 0 |
| | 2 | 0 | 0 |
| | 3 | 5.5 × 10⁵ | 8.0 × 10³ |
| | 4 | 0 | 0 |
| D | 1 | 0 | 0 |
| | 2 | 0 | 0 |
| | 3 | 0 | 0 |
| | 4 | 0 | 0 |
| E | 1 | 0 | 0 |
| | 2 | 0 | 0 |
| | 3 | 0 | 0 |
| | 4 | 0 | 0 |
| F | 1 | 1.5 × 10⁵ | 0 |
| | 2 | 4.5 × 10⁵ | 4.0 × 10³ |
| | 3 | 0 | 0 |
| | 4 | 5.0 × 10⁵ | 1.0 × 10⁴ |
| G | 1 | 0 | 0 |
| | 2 | 0 | 0 |
| | 3 | 0 | 0 |
| | 4 | 0 | 0 |
| H | 1 | 0 | 0 |
| | 2 | 0 | 0 |
| | 3 | 0 | 0 |
| | 4 | 0 | 0 |
| I | 1 | 1.9 × 10⁶ | 3.5 × 10⁴ |
| | 2 | 6.0 × 10⁵ | 7.5 × 10³ |
| | 3 | 4.0 × 10⁵ | 0 |
| | 4 | 0 | 0 |
| J | 1 | 2.3 × 10⁶ | 4.0 × 10⁴ |
| | 2 | 3.2 × 10⁶ | 4.5 × 10⁴ |
| | 3 | 2.5 × 10⁶ | 5.0 × 10⁴ |
| | 4 | 1.8 × 10⁶ | 2.0 × 10⁴ |

Summary

The experiment shows that colonization was abolished in groups dosed with cells (SP+VC) suspended in supernatant or PBS. SG57, SG137 and SG277 appeared to have similar efficacy. To evaluate the ability of B. amyloliquefaciens (Ba) strain SG277 to suppress or inhibit C. difficile infection (CDI) in vivo.

Study Plan

Species: Mouse C57 BL/6 (females, age 17-18 weeks) Source: Charles River. Housing: Animals were housed in groups (n=5) until the start of the study. After which they were housed individually in IVCs (independently ventilated cages). Food: Irradiated food pellets and sterile drinking water.

Test Products (TPs):

B. amyloliquefaciens SG277 in four forms or SG378 (negative control):
i) SG277 SP+VC in SUP

Cells were grown for 16 h at 37° C. in 25 ml BHIB. The cells were harvested from 100 ml culture (4 flasks) and the pellet resuspended in 2 ml of supernatant.

I dose=0.2 ml and ˜5×10⁹ CFU consisting of spores (˜70-90%)
ii) SG277 SP+VC in PBS

As above but the pellet was suspended in PBS

I dose=0.2 ml and ˜5×10⁹ CFU consisting of spores (˜70-90%)
iii) SG277 SUP
0.2 ml of supernatant from 16 h BHIB culture of SG277 supernatant was filter sterilised (0.45 μm) and used with 2 h
iv) SG277 spores

SG277 was grown on DSM agar trays for 72 h at 37° C. Spores crops (˜100% spores) were harvested and washed 3 times with sterile water and CFU of heat-resistant spores determined by serial dilutions and plating. Aliquots were prepared and stored at 4° C. till use.

I dose=0.2 ml and ˜5×10⁹ spores
v) SG378 SP+VC in SUP

Cells were grown for 16 h at 37° C. in 100 ml BHIB. The cells were harvested from 100 ml culture and the pellet resuspended in 2 ml of supernatant.

I dose=0.2 ml and ˜5×10⁹ CFU consisting of spores (˜70-90%)
B. difficile challenge, administration of the test composition and sampling were made as indicated in FIG. 2.

Challenge: CD630

Groups: n=10

| | |
|---|---|
| Gp A | SG277 SP + VC in SUP |
| Gp B | SG277 SP + VC in PBS |
| Gp C | SG277 SUP |
| Gp D | SG277 spores |
| Gp E | SG378 SP + VC in SUP |
| Gp F | 0.2 ml of PBS |

Analysis: Toxin A and B in caecum at 24 h

CFU of C. difficile spores in caecum at 24 h

Results

TABLE 4
Toxin A and B (ng/g) in caecum)

| | | Toxin A | Toxin B |
|---|---|---|---|
| Group | Mice | (ng/g) | (ng/g) |
| A | 1* | no sample | no sample |
| 277 vc + sp^{SUP} | 2 | 0 | 0 |
| | 3 | 0 | 0 |
| | 4 | 0 | 0 |
| | 5 | 0 | 0 |
| | 6 | 0 | 0 |
| | 7 | 0 | 0 |
| | 8 | 0 | 0 |
| | 9 | 0 | 0 |
| | 10 | 0 | 0 |
| B | 1 | 0 | 0 |
| 277 vc + sp^{PBS} | 2 | 0 | 0 |
| | 3 | 0 | 0 |
| | 4 | 0 | 0 |
| | 5 | 0 | 0 |
| | 6 | 0 | 0 |
| | 7 | 0 | 0 |
| | 8 | 0 | 0 |
| | 9 | 0 | 0 |
| | 10 | 45 | 0 |
| C | 1 | 0 | 0 |
| 277 SUP only | 2 | 0 | 0 |
| | 3 | 0 | 0 |
| | 4 | 0 | 0 |
| | 5 | 0 | 0 |
| | 6 | 365 | 186 |
| | 7 | 0 | 0 |
| | 8 | 0 | 0 |
| | 9 | 0 | 0 |
| | 10 | 0 | 0 |
| D | 1 | 593 | 861 |
| 277 spores | 2 | 1901 | 1466 |
| | 3 | 377 | 716 |
| | 4 | 1416 | 1426 |
| | 5 | 1290 | 1293 |
| | 6 | 2050 | 1505 |
| | 7 | 12 | 64 |
| | 8 | 0 | 0 |
| | 9 | 326 | 665 |
| | 10 | 917 | 949 |
| E | 1 | 103 | 0 |
| 378 vc + sp^{SUP} | 2 | 1191 | 1337 |
| | 3 | 533 | 1104 |
| | 4 | 262 | 850 |
| | 5 | 0 | 0 |
| | 6 | 902 | 1298 |
| | 7 | 1602 | 1438 |
| | 8 | 503 | 994 |
| | 9 | 1020 | 1181 |
| | 10 | 172 | 0 |
| F | 1 | 1669 | 1354 |
| Placebo | 2 | 1585 | 1482 |
| | 3 | 1380 | 1238 |
| | 4 | 653 | 757 |
| | 5 | 580 | 800 |
| | 6 | 1464 | 1070 |
| | 7 | 1655 | 1280 |
| | 8 | 317 | 451 |
| | 9 | 1451 | 1262 |
| | 10 | 1985 | 1253 |

*Mouse sick and had to be culled

TABLE 5
CFU in caecum

| | | CFU/g |
|---|---|---|
| Group | Mice | (EtOH Res) |
| A | 1* | no sample |
| 277 vc + sp^{SUP} | 2 | 0 |
| | 3 | 0 |
| | 4 | 0 |
| | 5 | 0 |
| | 6 | 0 |
| | 7 | 0 |
| | 8 | 0 |
| | 9 | 0 |
| | 10 | 0 |
| B | 1 | 0 |
| 277 vc + sp^{PBS} | 2 | 0 |
| | 3 | 0 |
| | 4 | 0 |
| | 5 | 0 |
| | 6 | 0 |
| | 7 | 0 |
| | 8 | 0 |
| | 9 | 0 |
| | 10 | 0 |
| C | 1 | 0 |
| 277 SUP only | 2 | 0 |
| | 3 | 0 |
| | 4 | 0 |
| | 5 | 0 |
| | 6 | 5.5 × 10⁴ |
| | 7 | 0 |
| | 8 | 0 |
| | 9 | 0 |
| | 10 | 0 |
| D | 1 | 6 × 10⁶ |
| 277 spores | 2 | 6.5 × 10⁶ |
| | 3 | 1.6 × 10⁶ |
| | 4 | 3.3 × 10⁶ |
| | 5 | 1.6 × 10⁷ |
| | 6 | 4.5 × 10⁶ |
| | 7 | 5 × 10⁵ |
| | 8 | 1.5 × 10⁴ |
| | 9 | 3.5 × 10⁴ |
| | 10 | 1 × 10⁶ |
| E | 1 | 5 × 10³ |
| 378 vc + sp^{SUP} | 2 | 1.2 × 10⁷ |
| | 3 | 5.5 × 10⁶ |
| | 4 | 8.5 × 10⁶ |
| | 5 | 5 × 10³ |
| | 6 | 7 × 10⁶ |
| | 7 | 7 × 10⁶ |
| | 8 | 5.5 × 10⁶ |
| | 9 | 7.5 × 10⁶ |
| | 10 | 1.5 × 10⁴ |
| F | 1 | 8.5 × 10⁶ |
| Placebo | 2 | 7 × 10⁶ |
| | 3 | 8 × 10⁶ |
| | 4 | 5.5 × 10⁶ |
| | 5 | 7 × 10⁵ |
| | 6 | 8 × 10⁶ |
| | 7 | 1.2 × 10⁷ |
| | 8 | 1 × 10⁶ |
| | 9 | 1 × 10⁷ |
| | 10 | 4.5 × 10⁶ |

*Mouse sick and had to be culled

Summary

These data provide strong evidence that a combination of spores and vegetative cells of Ba strain SG277 can provide protection against CDI.

### Example 4 Objective

To evaluate the ability of SG137 to suppress or inhibit Clostridioides difficile infection (CDI) in vivo.

Study Plan

Hamster (males, age 24 weeks) Source: Charles River. Housing: Animals were housed in groups until the start of the study, after which they were housed individually in IVCs (independently ventilated cages). Food: Irradiated food pellets and sterile drinking water.

Test Products

(TPs):
B. amyloliquefaciens SG137 grown 16 h at 37° C. in BHIB (25 ml) and pellet suspended in 0.6 ml of cell free supernatant. The suspension comprises spores (70-90%) and vegetative cells and is thus namedSP+VC Total CFU of 3×10¹⁰ in 0.2 ml/dose

Challenge: CD630 spores

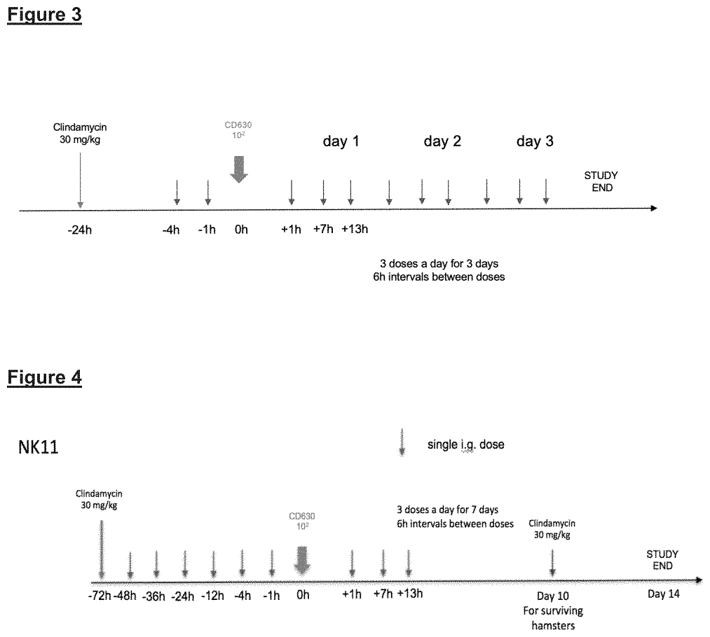

Regimen: FIG. 3

Dose: 0.2 ml i.g.

Groups: n=3

| | |
|---|---|
| Gp A | SG137 SP + VC in SUP |
| Gp B | Placebo (PBS buffer) |

Analysis: Survival Only

Results

TABLE 6
Survival data

| Time post challenge (h) | SG137 | placebo |
|---|---|---|
| 0 | 3/3 | 3/3 |
| 24 | 3/3 | 3/3 |
| 48 | 3/3 | 3/3 |
| 54 | 3/3 | 0/3 |
| 60 | 1/3 | 0/3 |
| 80 | 0/3 | 0/3 |

Summary

The experiment shows that administration of SG137 delayed symptoms in hamsters.

### Example 5 Objective

To evaluate the ability of SG137 and SG277 to suppress or inhibit Clostridioides difficile infection (CDI) in vivo.

Study Plan

Basically, the same as example 4 except:
(i) animals challenged 72 h post-clindamycin treatment rather than 24 h.
(ii) the cell pellet (SP+VC) was suspended in 6 ml of supernatant
(iii) 2 ml (3×10¹⁰ CFU) was used to dose each animal
(iv) hamsters at age ˜26 weeks

Dose: 2 ml i.g.

Group size: n=3

Dosing regime is shown in FIG. 4.

TABLE 7
Survival data

| time post challenge (h) | SG137 | SG277 | placebo |
|---|---|---|---|
| 0 | 3/3 | 3/3 | 3/3 |
| 24 | 3/3 | 3/3 | 3/3 |
| 48 | 2/3 | 3/3 | 1/3 |
| 72 | 2/3 | 3/3 | 0/3 |
| 79 | 1/3 | 3/3 | 0/3 |
| 96 | 1/3 | 3/3 | 0/3 |
| 103 | 0/3 | 3/3 | 0/3 |
| 120 | 0/3 | 3/3 | 0/3 |
| 144 | 0/3 | 3/3 | 0/3 |
| 168 | 0/3 | 3/3 | 0/3 |
| 240 | 0/3 | 1/3 | 0/3 |
| 336 | 0/3 | 1/3 | 0/3 |

Summary

SG277 showed clear evidence of protection and was superior to SG137.

### Example 6

Objective

To evaluate the ability of Bacillus amyloliquefaciens strain SG277 to suppress or inhibit Clostridioides difficile infection (CDI) in vivo.

Study Plan

Species: Mouse 057 BL/6 (females, age 10 weeks) Source: Envigo. Housing: Animals were housed in groups (n=5) until the start of the study. After which they were housed individually in IVCs (independently ventilated cages). Food: Irradiated food pellets and sterile drinking water.

Test Products (TPs):

B. amyloliquefaciens SG277 in four forms or PBS:

GP.1. SG277 SP+VC

Cells were grown for 16 h at 37° C. in 25 ml BHIB. The cells were harvested from 100 ml culture (4 flasks) and the pellet resuspended in 2 ml of PBS.

$$1 dose = 0.2 ml and \sim 5 \times {10}^{9} CFU consisting of spores ( \sim 70 \text{-} 100 \% )$$

$$Gp .2 . Freeze \text{-} dried SG 277 SP + VC ( SG 277 SP + {VC}^{FD} )$$

Cells were grown for 16 h at 37° C. in 25 ml BHIB. Cells pellet was then frozen at −20° C. o/n. The next day tubes containing frozen pellet from 25 ml culture were covered with perforated parafilm and lyophilized o/n. When ready, stored at −20° C. (lids on, parafilmed). On the day of use, pellet from 4 flasks was harvested and resuspended in 2 ml of PBS.

1 dose=0.2 ml and ˜5×10⁹ CFU consisting of spores (˜70-100%)

GP.3. SG277 SUP

1 dose=0.2 ml of filtered supernatant from 16 h BHIB culture of SG277

GP.4. SG277 SEC

Supernatant from a 16 h BHIB culture of SG277 was filtered and then processed first by Ammonium sulphate precipitation and then by size exclusion chromatography (SEC). By SEC, the “active” fractions were assessed and isolated for use in this experiment. According to the purification protocol and water was used as final vehicle. Samples aliquots were stored at −20° C. and thawed on the day of use.

1 dose=0.2 ml of SEC sample

GP.5. Placebo

1 dose=0.2 ml of PBS

Challenge: 1×10² of CD630 spores

Regimen: FIG. 5

Groups: n=8

| | |
|---|---|
| Gp 1 | SG277 SP + VC |
| Gp 2 | SG277 SP + VC^{FD} |
| Gp 3 | SG277 SUP |
| Gp 4 | SG277 SEC |
| Gp 5 | Placebo (PBS) |

Analysis: Toxin A and B in cecum at 24 h (SOP27) CFU of C. difficile spores in cecum at 24 h (SOP26).

Results

TABLE 8
Toxin A and B (ng/g) in cecum

| | | Toxin A | Toxin B |
|---|---|---|---|
| Group | Mice | (ng/g) | (ng/g) |
| SG277 SP + VC | 1 | 0 | 0 |
| | 2 | 0 | 0 |
| | 3 | 0 | 0 |
| | 4 | 0 | 0 |
| | 5 | 0 | 0 |
| | 6 | 0 | 0 |
| | 7 | 0 | 0 |
| | 8 | 0 | 0 |
| SG277 SP + VC^{FD} | 1 | 0 | 0 |
| | 2 | 0 | 0 |
| | 3 | 0 | 0 |
| | 4 | 0 | 0 |
| | 5 | 0 | 0 |
| | 6 | 0 | 0 |
| | 7 | 0 | 0 |
| | 8 | 0 | 0 |
| SG277 SUP | 1 | 0 | 0 |
| | 2 | 0 | 0 |
| | 3 | 0 | 0 |
| | 4 | 0 | 0 |
| | 5 | 0 | 0 |
| | 6 | 0 | 0 |
| | 7 | 0 | 0 |
| | 8 | 0 | 0 |
| SG277 SEC | 1 | 0 | 124 |
| | 2 | 0 | 170 |
| | 3 | 0 | 134 |
| | 4 | 0 | 0 |
| | 5 | 0 | 0 |
| | 6 | 0 | 51 |
| | 7 | 257 | 352 |
| | 8 | 0 | 0 |
| Placebo | 1 | 976 | 792 |
| | 2 | 1102 | 653 |
| | 3 | 743 | 603 |
| | 4 | 1201 | 812 |
| | 5 | 1136 | 666 |
| | 6 | 1481 | 589 |
| | 7 | 1287 | 538 |
| | 8 | 1334 | 806 |

TABLE 9
CFU in cecum

| | | CFU/g |
|---|---|---|
| Group | Mice | (EtOH Res) |
| SG277 SP + VC | 1 | 0 |
| | 2 | 0 |
| | 3 | 0 |
| | 4 | 0 |
| | 5 | 0 |
| | 6 | 0 |
| | 7 | 0 |
| | 8 | 0 |
| SG277 SP + VC^{FD} | 1 | 0 |
| | 2 | 0 |
| | 3 | 0 |
| | 4 | 0 |
| | 5 | 0 |
| | 6 | 0 |
| | 7 | 0 |
| | 8 | 0 |
| SG277 SUP | 1 | 0 |
| | 2 | 1.85 × 10⁵ |
| | 3 | 0 |
| | 4 | 0 |
| | 5 | 0 |
| | 6 | 0 |
| | 7 | 3 × 10³ |
| | 8 | 0 |
| SG277 SEC | 1 | 1.04 × 10⁵ |
| | 2 | 1 × 10⁵ |
| | 3 | 8.5 × 10⁴ |
| | 4 | 0 |
| | 5 | 0 |
| | 6 | 5 × 10⁵ |
| | 7 | 1 × 10⁶ |
| | 8 | 2.5 × 10⁵ |
| Naive | 1 | 7 × 10⁶ |
| | 2 | 3.4 × 10⁶ |
| | 3 | 7 × 10⁶ |
| | 4 | 1 × 10⁷ |
| | 5 | 5.5 × 10⁶ |
| | 6 | 6.5 × 10⁶ |
| | 7 | 1 × 10⁷ |
| | 8 | 8.25 × 10⁶ |

Summary

Administration of ˜5×10⁹ CFU of freeze-dried cells of B. amyloliquefaciens SG277 (Gp.2) was efficient in suppressing CDI in a murine model. No toxin A and B levels were detected in caeca of mice infected with CD630. Bacteria remain viable after lyophilisation process and they retain their antimicrobial activity.

Administration of SG277 supernatant active compound purified through liquid chromatography (Gp.4) significantly decreased toxins levels in caeca detected at 24 h post-challenge. In ⅜ samples toxins were not detected.

### Example 7

Objective

To evaluate the ability of N-acetyl-glucosamine (GlcNAc) or glucosamine to suppress or inhibit Clostridioides difficile infection (CDI) in animals.

Design: Holloway model of CDI. C₅₇BL/6 (age 9 weeks). Six (6) mice per group were housed in independently ventilated cages. Animals were dosed with test products (TPs) ad libitum starting 2 days before clindamycin treatment and then for the duration of the study. Infection started at hour 0 with oral dosing (intra-gastric gavage) of clindamycin (30 mg/kg) and then at 24 h animals were challenged with 100 pure spores of CD strain 630. Animals were monitored for 24 h post challenge after which caeca were removed and examined for levels of Toxin A and Toxin B.
Groups

| | |
|---|---|
| Gp1 | GlcNAc 20 mg/ml in drinking water |
| Gp2 | GlcNAc 50 mg/ml in drinking water |
| Gp3 | GlcNAc 100 mg/ml in drinking water |
| Gp4 | Glucosamine 20 mg/ml in drinking water |
| Gp5 | Glucosamine 50 mg/ml in drinking water |
| Gp6 | Glucosamine 100 mg/ml in drinking water |
| Gp7 | water only |

GlcNAc (Sigma A4106). Glucosamine hydrochloride (Sigma G4875)

TABLE 10
Water consumption¹

| | | | | | Daily | Daily dose |
|---|---|---|---|---|---|---|
| | | | | | con- | of TP |
| | Initial | Left- | | Daily con- | sumption | adminis- |
| | volume | over | | sumption | per | tered |
| | given | volume | [symbol] erence | per cage | mouse | to mouse |
| Group | (ml) | (ml) | (ml) | (ml, n = 6) | (ml) | (mg) |
| Gp 1 | 200 | 145 | 55 | 18.3 | 3 | 60 |
| Gp 2 | 200 | 150 | 50 | 16.7 | 2.8 | 140 |
| Gp 3 | 200 | 155 | 45 | 15 | 2.5 | 250 |
| Gp 4 | 200 | 125 | 75 | 25 | 4.2 | 84 |
| Gp 5 | 200 | 150 | 50 | 16.7 | 2.8 | 140 |
| Gp 6 | 200 | 180 | 20 | 6.7 | 1 | 100 |
| Gp 7 | 200 | 150 | 50 | 16.7 | 2.8 | 0 |

Observations: Animals in Gp6 appeared dehydrated and drank less water (Table 10). Other animals looked healthy. [symbol]
indicates data missing or illegible when filed

TABLE 11
Toxins raw data

| | | Toxin A | Toxin B |
|---|---|---|---|
| Group | Mouse | (ng/g) | (ng/g) |
| GP1 | 1 | 244.93 | 21.34 |
| | 2 | 206.52 | 53.47 |
| | 3 | 138.16 | 0.00 |
| | 4 | 43.68 | 0.00 |
| | 5 | 2359.98 | 578.85 |
| | 6 | 132.01 | 43.45 |
| GP2 | 1 | 94.76 | 69.74 |
| | 2 | 113.58 | 33.02 |
| | 3 | 117.03 | 6.31 |
| | 4 | 435.04 | 129.42 |
| | 5 | 0.00 | 0.00 |
| | 6 | 0.28 | 0.00 |
| GP3 | 1 | 89.76 | 3.81 |
| | 2 | 100.13 | 0.00 |
| | 3 | 49.44 | 64.32 |
| | 4 | 200.37 | 128.58 |
| | 5 | 142.76 | 37.19 |
| | 6 | 151.98 | 76.00 |
| GP4 | 1 | 266.05 | 61.40 |
| | 2 | 119.34 | 132.34 |
| | 3 | 226.49 | 131.50 |
| | 4 | 208.82 | 73.50 |
| | 5 | 1152.09 | 698.61 |
| | 6 | 216.51 | 56.81 |
| GP5 | 1 | 564.85 | 265.04 |
| | 2 | 6.04 | 0.00 |
| | 3 | 264.51 | 72.25 |
| | 4 | 158.13 | 14.24 |
| | 5 | 236.86 | 0.00 |
| | 6 | 292.55 | 0.00 |
| GP6 | 1 | 129.32 | 29.68 |
| | 2 | 160.43 | 23.01 |
| | 3 | 787.99 | 201.61 |
| | 4 | 161.97 | 0.00 |
| | 5 | 221.88 | 0.00 |
| | 6 | 166.19 | 24.68 |
| GP7 | 1 | 1169.37 | 507.91 |
| | 2 | 1053.00 | 374.79 |
| | 3 | 903.98 | 274.64 |
| | 4 | 873.26 | 145.69 |
| | 5 | 831.39 | 186.59 |
| | 6 | 638.98 | 590.95 |

TABLE 12
Ethanol resistant CFU Raw data

| Group | Mouse | CFU/g faeces |
|---|---|---|
| GP1 | 1 | 1.45E+05 |
| | 2 | 0.00E+00 |
| | 3 | 0.00E+00 |
| | 4 | 0.00E+00 |
| | 5 | 9.00E+05 |
| | 6 | 0.00E+00 |
| GP2 | 1 | 0.00E+00 |
| | 2 | 0.00E+00 |
| | 3 | 0.00E+00 |
| | 4 | 0.00E+00 |
| | 5 | 0.00E+00 |
| | 6 | 0.00E+00 |
| GP3 | 1 | 0.00E+00 |
| | 2 | 0.00E+00 |
| | 3 | 0.00E+00 |
| | 4 | 0.00E+00 |
| | 5 | 0.00E+00 |
| | 6 | 0.00E+00 |
| GP4 | 1 | 0.00E+00 |
| | 2 | 0.00E+00 |
| | 3 | 0.00E+00 |
| | 4 | 0.00E+00 |
| | 5 | 1.90E+06 |
| | 6 | 0.00E+00 |
| GP5 | 1 | 0.00E+00 |
| | | 0.00E+00 |
| | 3 | 0.00E+00 |
| | 4 | 0.00E+00 |
| | 5 | 0.00E+00 |
| | 6 | 0.00E+00 |
| GP6 | 1 | 0.00E+00 |
| | 2 | 0.00E+00 |
| | 3 | 1.70E+05 |
| | 4 | 0.00E+00 |
| | 5 | 8.00E+04 |
| | 6 | 0.00E+00 |
| GP7 | 1 | 7.50E+06 |
| | 2 | 4.00E+06 |
| | 3 | 3.60E+06 |
| | 4 | 1.55E+06 |
| | 5 | 1.45E+06 |
| | 6 | 9.50E+05 |

Summary

Administration of GlcNAc or glucosamine at levels as low as 20 mg/ml in drinking water could suppress CD colonisation.

Conclusion

Water containing 20 mg/ml of GlcNAc or glucosamine hydrochloride prevented CD infections (CDI) in mice. This equates to a mouse consuming approximately 3 g/kg/day.

### Example 8

Objective

Characterization of the Antimicrobial Activity of Bacterial Strains

### Preparation of Cell Free Filtrates

Bacillus strains were grown in BHIB (Brain heart infusion broth, Oxoid CM1135) medium grown at 37° C. O/N under aerobic condition (by agitation). The cultures were centrifuged (8,000 g for 15 min.) and the supernatant filter-sterilized using 0.45 m filters. The filtrates were stored on ice and used the day they were produced. Alternatively, Aerobic Bacillus strains were grown in LB medium at 37° C. for 16-18 h and supernatants filter-sterilized (0.20 μm syringe filter) and stored on ice till use. For Clostridioides difficile TGY agar plates were pre-reduced (4 h in an anaerobic chamber) (TGY agar is, per litre, tryptic soy broth (30 g), glucose (20 g), yeast extract (10 g), L-cysteine (1 g), Resazurin (1 mg) and agar (15 g)). Plates were then spread with an overnight C. difficile culture (˜100 μl) and allowed to dry for 30 min. after which 4-6 wells (5 mm) were cut per plate. 50 μl of Bacillus supernatants was applied to labelled wells and the plates incubated at 37° C. for 48 h in an anaerobic chamber and zones of inhibition measured (diameter), typically 9-20 mm.

| | CD strain | | | | |
|---|---|---|---|---|---|
| | CD630 | CD196 | R20291 | VPI 10463 | SH1 |
| Test | rt012 | rt027 | rt027 | rt087 | rt078 |
| SG277 | 26 ± 0 | 26 ± 1.4 | 28.5 ± 2.1 | 27.5 ± 3.5 | 26.5 ± 4.9 |
| SG297 | 26 ± 1.4 | 24.5 ± 0.7 | 23 ± 2.8 | 25 ± 0 | 25.5 ± 0.7 |

### Preparation of Indicator Culture

A single colony of the relevant C. difficile strain was inoculated into 10 ml of BHIS and incubated overnight at 37° C. in an anaerobic chamber. The overnight culture was then sub-cultured 1:100 into BHIS (typically 0.1 ml into 10 ml BHIS) and incubated at 37° C. for 6 h after which the culture was ready for use. BHIS (Brain Heart Infusion supplemented with yeast extract and L-cysteine) broth: 37 g Brain heart infusion (Oxoid CM1135), 5 g Yeast extract (Oxoid LP0021), 1 g L-cysteine (Sigma-Aldrich C7352) 1000 μl 1000× Resazurin (Sigma-Aldrich R7017) dH₂0 to 1000 ml

Plate Set Up

180 μl of sterile BHIS was pipetted into the first row of a 96-well U-bottom microplate (Sigma) and 100 μl into each subsequent row. 20 μl of the sample to be tested was pipetted into the first row (1:10 dilution factor) and serially diluted in a 2-fold dilution series until the last row (1:1280 dilution factor) on the microplate. For one serial dilution a media-only control was also pipetted into a single well on the first column. 10 μl of the 6 h C. difficile ‘indicator culture’ was pipetted into each well and the plate was incubated overnight at 37° C. in an anaerobic chamber. After overnight growth the microplate contents were agitated on a rotary plate shaker at 200 rpm for 2 min. after which the OD₆₀₀ was measured using a microplate plate reader. Positive inhibitory activity was defined as an OD₆₀₀ increase of <50% of the media-only control. Antimicrobial activity measured with the microdilution assay to quantify the level of extracellular inhibitory activity against C. difficile strain CD630:

| | | Endpoint titre² | | |
|---|---|---|---|---|
| Strain | Species | expt 1 | expt 2 | expt 3 |
| PY79 | subtilis | 0 | 0 | 0 |
| SG140 | subtilis | 1/20 | 1/20 | 1/10 |
| 10A1 | amyloliquefaciens | 0 | 0 | 0 |
| SG57 | amyloliquefaciens | 1/40 | 1/40 | 1/40 |
| SG137 | amyloliquefaciens | 1/40 | 1/40 | 1/40 |
| SG185 | amyloliquefaciens | 1/80 | 1/40 | 1/40 |
| SG277 | amyloliquefaciens | 1/160 | 1/160 | 1/80 |
| SG297 | amyloliquefaciens | 1/160 | 1/320 | 1/160 |

B. subtilis PY79 B. subtilis and B. amyloliquefaciens 10A1 were control strains obtained from the Bacillus Genetic Stock Centre (BGSC).
C. difficile cultures were grown in BHIS for 10 h at 37° C. 180 μl of the C. difficile culture was added to a microplate well followed by 20 μl of a sterile SG277 filtrate. Plates were incubated anaerobically 18 h at 37° C. after which the OD600 was read. The SG277 filtrate was also incubated overnight as a control and showed no growth.

| | |
|---|---|
| C. difficile | OD₆₀₀ |

| strain | Ribotype | Untreated | +SG277 | % lysis |
|---|---|---|---|---|
| CD630 | RT012 | 0.756 | 0.059 | 92 |
| SH1 | RT078 | 0.845 | 0.086 | 90 |
| SH101 | RT115 | 0.772 | 0.081 | 89.5 |
| SH102 | RT176 | 0.981 | 0.072 | 93 |
| R20291 | RT027 | 0.857 | 0.091 | 89 |
| CD196 | RT027 | 0.798 | 0.056 | 93 |
| SH104 | RT023 | 0.534 | 0.056 | 89.5 |
| VPI 10463 | RT087 | 0.687 | 0.054 | 92 |
| CD10 | non-tox | 0.824 | 0.102 | 88 |
| SH242 | RT111 | 0.672 | 0.089 | 87 |
| SH200 | RT056 | 0.914 | 0.076 | 92 |
| SH203 | RT038 | 0.882 | 0.064 | 93 |
| SH218 | RT001 | 0.732 | 0.081 | 89 |
| SH210 | RT002 | 0.655 | 0.055 | 92 |
| SH213 | RT014 | 0.758 | 0.064 | 92 |
| SH215 | RT54 | 1.025 | 0.056 | 95 |
| SH220 | RT336 | 0.783 | 0.049 | 94 |
| SH222 | RT401 | 0.952 | 0.082 | 91 |
| SH231 | RT56 | 0.791 | 0.073 | 91 |
| SH236 | RT005 | 0.883 | 0.091 | 90 |
| SH239 | RT103 | 0.966 | 0.086 | 91 |
| SH103 | RT075 | 0.761 | 0.106 | 86 |
| SH3 | RT017 | 0.692 | 0.055 | 92 |
| SH1 | RT005 | 0.883 | 0.091 | 90 |

Isolation and Characterization of Bioactive Compounds Bacterial Growth

SG277 was streaked on DSM agar, incubated O/N at 37° C. and a single colony used to inoculate 25 ml of BHIB broth. The culture was allowed to grow for 16 h at 37° C. The culture was centrifuged to pellet bacteria and the supernatant removed after centrifugation and filtered through a 0.45 m membrane. Stored on ice for up to 4 h or frozen at −20° C.

Fractionation

Using centrifugal concentrators of different molecular weight cut-offs (MWCO) we determined the approximate mwt. of Amycide™ contained within the filter-sterilised (0.45 μm) supernatant from SG277 (Table 13). Activity (determined using the microdilution assay) was found in two fractions: 30-100 kDa and >100 kDa, suggesting that Amycide™ might exist as a complex, be physically labile and could dissociate while retaining some activity.

Sterile filtered (0.45 μm) culture supernatant were fractionated using Vivaspin 6 centrifugal concentrators at cut-off indicated in the table. Fractions were tested for activity against CD630 measured using microdilution assay.

TABLE 13

| Cut-off (kDa) | Activity titre |
|---|---|
| Untreated | 1/80 |
| <10 | 0 |
| 10-30 | 0 |
| 30-100 | 1/40 |
| >100 | 1/40 |

Ammonia Sulphate Precipitation

AmSO₄ (113 g/L) was added to the sterile filtrate to give a 20% w/v solution and incubated O/N at 4° C. The solution was then centrifuged, and the pellet was suspended in PBS at a concentration of 30× (that is, for 30 ml of initial culture the ppt would be dissolved in 1 ml of PBS). The Am—SO₄ ppt was dialysed in PBS O/N (at 4° C.) to remove excess AmSO₄. Activity of AmSO₄ ppt=1/2560. This method enabled precipitation of the large molecular weight species responsible for the functional activity (as evidenced in MWCO experiments), and to reduce the amount of protein which would be co-purified alongside them.

Preparative SEC¹ Separation

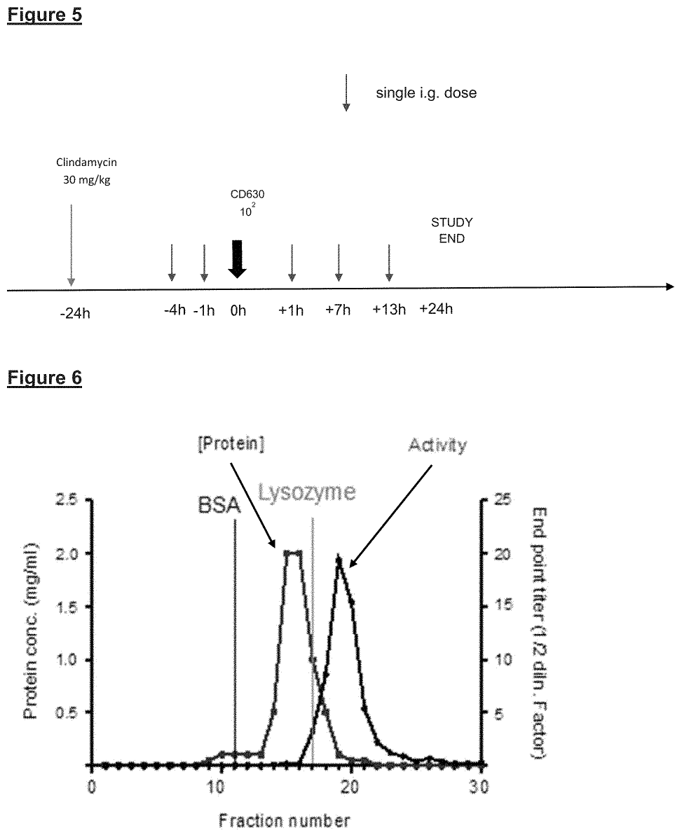

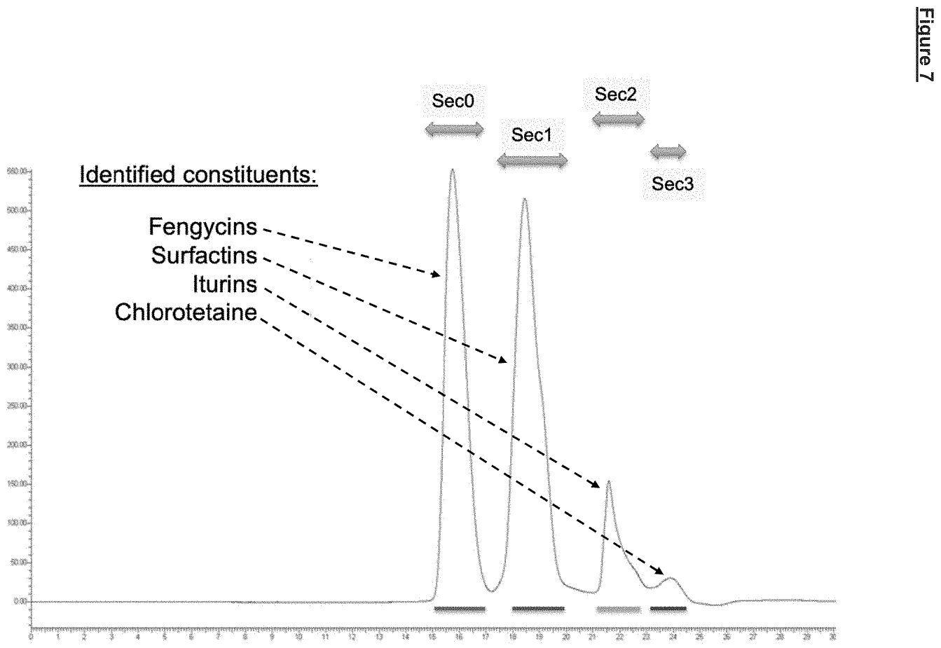

In order to further purify the active species, the AmSO₄ precipitate was separated using a Superdex 200 column (10,000 Da-600,000 Da) in PBS+0.1% SDS (w/v). This allowed a preparative separation of high MW species. SDS was added to denature/linearize unwanted proteins resulting in a purer separation. Fractions were tested for activity against CD630 (using dilution factor or endpoint titre) using a microplate assay and active fractions were combined and washed to remove excess SDS. This analysis revealed that anti-CD630 activity did not correlate with protein content. Using the in vitro assay for activity the SEC fraction gave an endpoint titre of 1/5120 (as illustrated in FIG. 6: internal markers (BSA and lysozyme) run on the same column).

HPLC Analysis

To examine further the constituent species of the preparative SEC active fraction, separation was performed using HPLC using a column that separated molecules in the range of 200-3,000 Da. Acetonitrile was used as the isocratic buffer (FIG. 7).

The four HPLC fractions were tested for activity against CD630, all four fractions showed activity

| Fraction | Composition | anti-CD activity |
|---|---|---|
| Sec0 | fengycins | 1/10 |
| Sec1 | surfactins | 1/320 |
| Sec2 | iturins | 1/10 |
| Sec3 | chlorotetaine | 1/40 |

Bioinformatics Analysis

By Bioinformatics analysis of the whole genome sequence genes encoding enzymes involved in the biosynthesis of the non-ribosomal peptides fengycin, surfactin and chlorotetaine were found in all six strains tested 297. For example, the Malonyl CoA-acyl carrier protein transacylase from SG277 gene (Seq ID NO 9 and Seq ID NO 10) encoded by the genes Seq ID NO 7 and Seq ID NO 8.

Conclusion

In the Bioinformatics analysis, the genetic basis of fengycin, surfactin and chlorotetaine was demonstrated in all six strains and iturin was found in SG277 and SG297.

### DEPOSIT OF BIOLOGICAL MATERIAL

The following biological material has been deposited under the terms of the Budapest Treaty with the NCIMB, Ferguson Building, Crabstone Estate, Bucksburn, Aberdeen, AB21 9YA, and given the following accession number:

| Deposit | Accession Number | Date of Deposit |
|---|---|---|
| B. amyloliquefaciens SG277 | NCIMB 42971 | 15 Feb. 2018 |
| B. amyloliquefaciens SG297 | NCIMB 42972 | 15 Feb. 2018 |
| B. amyloliquefaciens SG185 | NCIMB 42973 | 15 Feb. 2018 |
| B. subtilis SG140 | NCIMB 42974 | 15 Feb. 2018 |
| B. amyloliquefaciens SG57 | NCIMB 43392 | 10 May 2019 |
| B. amyloliquefaciens SG137 | NCIMB 43393 | 10 May 2019 |

The strains have been deposited under conditions that assure that access to the culture will be available during the pendency of this patent application to one determined by foreign patent laws to be entitled thereto. The deposits represent a substantially pure culture of the deposited strain. The deposits are available as required by foreign patent laws in countries wherein counterparts of the subject application, or its progeny are filed. However, it should be understood that the availability of a deposit does not constitute a license to practice the subject invention in derogation of patent rights granted by governmental action.

1. A composition comprising one or more microorganisms selected from Bacillus amyloliquefaciens and Bacillus subtilis strains, and/or extracellular material produced by one or more of the microorganisms, and one or more food grade ingredients, wherein the microorganism has antimicrobial activity against pathogenic strains of Clostridioides difficile and produces two or more non-ribosomal peptides.

2. The composition of claim 1, wherein the two or more non-ribosomal peptides are selected from the group consisting of: a member of the Fengycin family; a member of the Surfactin family; a member of the Iturin family; and Chlorotetaine.

3. The composition of claim 1 or 2, wherein the two or more non-ribosomal peptides are selected from the group consisting of: a member of the Fengycin family; a member of the Surfactin family; and Chlorotetaine.

4. The composition of any preceding claim, wherein the microorganism produces three or more non-ribosomal peptides, optionally wherein the non-ribosomal peptides are: a member of the Fengycin family; a member of the Surfactin family; and Chlorotetaine.

5. The composition according to any one of the preceding claims, wherein the microorganism comprises a gene with at least 90% sequence identity to SEQ ID NO: 7 or at least 90% sequence identity to SEQ ID NO: 8, such as at least 95% sequence identity to SEQ ID NO: 7, at least 95% sequence identity to SEQ ID NO: 8, at least 98% sequence identity to SEQ ID NO: 7, at least 98% sequence identity to SEQ ID NO: 8, at least 99% sequence identity to SEQ ID NO: 7, at least 99% sequence identity to SEQ ID NO: 8, 100% sequence identity to SEQ ID NO: 7 or 100% sequence identity to SEQ ID NO: 8.

6. The composition according to any one of the preceding claims, wherein the microorganism comprises a gene coding for a protein sequence with at least 90% sequence identity to SEQ ID NO: 9 or at least 90% sequence identity to SEQ ID NO: 10, such as at least 95% sequence identity to SEQ ID NO: 9, at least 95% sequence identity to SEQ ID NO: 10, at least 98% sequence identity to SEQ ID NO: 9, at least 98% sequence identity to SEQ ID NO: 10, at least 99% sequence identity to SEQ ID NO: 9, at least 99% sequence identity to SEQ ID NO: 10, 100% sequence identity to SEQ ID NO: 9 or 100% sequence identity to SEQ ID NO: 10.

7. The composition according to any one of the preceding claims, wherein the microorganism comprises 16S rDNA that is more than 98% sequence identity, such as more than 98% sequence identity or 100% sequence identity, to SEQ ID NO: 1 and/or more than 98% sequence identity such as more than 98% sequence identity or 100% sequence identity, to SEQ ID NO: 2 and/or more than 98% sequence identity such as more than 98% sequence identity or 100% sequence identity, to SEQ ID NO: 3 and/or more than 98% sequence identity such as more than 98% sequence identity or 100% sequence identity, to SEQ ID NO: 4 and/or more than 98% sequence identity such as more than 98% sequence identity or 100% sequence identity, to SEQ ID NO: 5 and/or more than 98% sequence identity such as more than 98% sequence identity or 100% sequence identity, to SEQ ID NO: 6.

8. The composition according to any one of the preceding claims, wherein the composition comprises a Bacillus amyloliquefaciens strain, and the Bacillus amyloliquefaciens strain is selected from the strains deposited as NCIMB 42971, NCIMB 42972, NCIMB 42973, NCIMB 43392 or NCIMB 43393.

9. The composition according to any one of the preceding claims, wherein the composition comprises a Bacillus subtilis strain, and the Bacillus subtilis strain is the strain deposited as NCIMB 42974.

10. The composition according to any one of the preceding claims, wherein all of the one or more microorganisms are selected from the Bacillus amyloliquefaciens strains deposited as NCIMB 42971, NCIMB 42972, NCIMB 42973, NCIMB 43392 or NCIMB 43393; and the Bacillus subtilis strain deposited as NCIMB 42974.

11. The composition according to any one of the preceding claims, being a food, food ingredient, dietary supplement, dietary supplement ingredient, medical food, food for special medical purposes, food for specified health use, food for special dietary use, health food, Complementary Medicine; Natural Health Product, Natural Health formulation, Natural Health ingredient, pharmaceutical product, pharmaceutical preparation, pharmaceutical formulation, or a pharmaceutical ingredient.

12. The composition according to claim 11, wherein the composition is a probiotic composition comprising or consisting of live or dead cells, or spores, or compounds originating from the cells or spores.

13. The composition according to claim 12, wherein the composition comprises one or more food grade ingredients selected from fillers and stabilizing agents.

14. The composition according to any of claims 11 or 12, wherein the composition is provided in a unit dosage formulation, such as a capsule, tablet or sachet.

15. The composition of claim 14, wherein each unit dosage formulation comprises 10⁸ to 10¹⁰ CFU of the one or more microorganisms.

16. The composition of claim 11, wherein the composition is a food composition, comprising at least one nutrient and/or vitamin in addition to the one or more microbial strains.

17. The composition of claim 16, wherein the food composition comprises a CFU count corresponding to 10⁸ to 10¹⁰ CFU per serving.

18. The composition according to any one of the preceding claims, wherein the one or more microorganisms is provided in lyophilized or spray-dried form.

19. The composition according to any one of the preceding claims, further comprising glucosamine.

20. The composition of claim 19, wherein the composition comprises glucosamine corresponding to a daily dosage of 10-2000 mg, optionally in the range of 100-2000 mg, or in the range of 250-1500 mg, or in the range of 500-1000 mg.

21. The composition of either claim 19 or claim 20, wherein the glucosamine is glucosamine hydrochloride or glucosamine phosphate.

22. A use of the composition according to any of the preceding claims, for the prevention of C. difficile infection in an individual at risk of obtaining such an infection, or reduction of duration or severity of a C. difficile infection, or prevention or reduction of asymptomatic C. difficile colonization.

23. The use of claim 22, wherein 10⁸ to 10¹⁰ CFU Bacillus cells are administered daily.

24. The use of either claim 22 or 23, wherein the composition prevents or reduces C. difficile infection in the gastrointestinal tract of a subject, preferably a human subject.

25. The composition according to any one of claims 1 to 21, for use in the prevention of C. difficile infection in an individual at risk of obtaining such an infection, or reduction of duration or severity of a C. difficile infection, or prevention or reduction of asymptomatic C. difficile colonization.


SEQUENCE LISTING (27 sequences)

SEQ ID NO: 1 - DNA, 1555 bp, Bacillus amyloliquefaciens
tttatcggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtctgaaccgcatggttcagacatgaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctcctttctaa

SEQ ID NO: 2 - DNA, 1555 bp, Bacillus amyloliquefaciens
tttatcggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactacgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctcctttctaa

SEQ ID NO: 3 - DNA, 1555 bp, Bacillus amyloliquefaciens
tttatcggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctcctttctaa

SEQ ID NO: 4 - DNA, 1273 bp, Bacillus subtilis
acgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctcctttctaagga

SEQ ID NO: 5 - DNA, 1555 bp, Bacillus amyloliquefaciens; identical to SEQ ID NO: 3

SEQ ID NO: 6 - DNA, 1555 bp, Bacillus amyloliquefaciens; identical to SEQ ID NO: 3

SEQ ID NO: 7 - DNA, 1203 bp, Bacillus amyloliquefaciens
atgaacaatcttgcctttttatttcctggacaagggtctcaatttgtaggaatgggcaaacaattttggaatgattttgtgctcgcaaagagattgtttgaagaagcgagcgatgcgatctccttggatgtaaaaaaactgtgttttaacggagatatgaatgaattgacaaagacaatgaacgcgcagcccgctattttaacggtcagtgttattgcttttcaagtgtatatgcaggaaataggggtgaagccccgcttcctggcaggccatagcttaggcgaatattcagcgcttgtctgtgccggcgccctttcttttcaggatgccgttacacttgtaaggcagcggggaattcttatgcagaatgcggatcctcagcagcaggggacgatggccgccgtgactcacctctctcttcaaacgttgcaggaaatatgttcgaaagtgtcgacggaagactttccggcaggagtagcctgcatgaattcagaacagcagcatgtgatttccggacaccggcaagctgtggaacgtgtcatcaagatggcggaggaaaagggagcggcatacacttatttgaatgtcagtgcgccttttcacagttcgctgatacgatcagcatctgaacaattccagactgtattacaccggtattccttccgggatgccgcatggccgatcatttcaaatgtcaccgcacgcccttacagcagcggaaattcaatcagcgaacatctcgagcagcacatgacgatgccggtaagatggacggaatcgatgcattacttgcttttacacggagtcacagaagtcatcgaaatgggtccgaacaatgtcttagccggtctgctgagaaaaacaacgaatcacattgtaccttatcccttaggacagacatctgatgttcacttgctttccaattcagcagaaagaaagaaacatattgtccgtttacgcaaaaaacaactgaataaattgatgattcaatccgtcattgcgcgaaattacaacaaggattcagcggcttattccaatatgacgacggcattatttacgcaaatccaagagctgaaagagagaatggaaagacatgaaaatgagctctcagaacaagagctcgaacattcgatccatttatgcaaattaatttgcgaggctaaacagcttccggattgggaagaattgcggattttaaaataa

SEQ ID NO: 8 - DNA, 11949 bp, Bacillus amyloliquefaciens
atgtataccagtcaattccaaaccttagtagatgtcattcgggaaagaagcaatatctctgaccgcgggatccgttttatcgaatccgataaaaacgagacggttgtctcttatcgccaattgtttgaagaggcgcaagggtatcttggctatttacagcatatcggcattcagccgaagcaggaaattgtatttcaaatccaagaaaacaaatcatttgtcgttgctttttgggcttgtatattaggaggaatgatcccggtgccggtcagtatcggagaagatgatgaccataagctgaaggtctggcgcatttggaatatattaaatcacccgtttctgattgcctctgaaaaagtattggacaaaataaagaaatacgctgcagaacacgatttacaggatttccatcatcaattaaacgaaaaatctgacatcattcaagatcaaacctacgattaccccgcttcgttttatgaacctgatgcggatgaactcgcctttatccaattttcttcaggatcgacaggagatccaaaaggagtcatgttaacgcatcacaacttaatacataacacgtgcgccattgggaatgccctagccgttcattcgagagactctttcttatcatggatgcctttaacgcatgatatggggctcatcgcctgccaccttgttcccttcataaccggaatcaatcaaaatctgatgcctacagaattatttattcgcagacctattctttggatgaaaaaagctcatgaacataaagccagtattctatcctctcctaatttcggatacaactacttccttaaatttctgaaaaacgaaccagactgggatttatcccacatcaaggtcatcgcaaacggtgcagaaccgatattgccggagctctgtgacgaatttttgaaaagatgcgcagcattcaatctgaaaagatccgccattttgaatgtttacggtttagcggaagcttcggtcggcgcagcattctctaaattaggtaaagaattcgttcccgtttatttgcatcgcgatcatttaaatctcggtgaaagagctgtaaacgtcagcaaagaggatcaaaattgcgcttcattcgtcgaagtgggacgacctattgactattgtcagcttcggatctccgatgaagcaaatgaaagagtagaagacggaatcatcggccatatccagatcaaaggagacaatgtgactcaagggtattataacaaccccgagagtacggaaaaagcgctgacttctgacggctgggtaaaaacgggagacctcggattcattagtgaaagtggtaacttagtcgtaaccggaagagaaaaggacattattttcgtgaacggaaaaaatatctacccgcacgatattgaacgggtagcgattgaaatggaagaggttgacttaggaagggttgccgcctgcggtgtatatgatcaaaagacacaaagcggagaaatcgtgctctttgttgtttacaaaaaatcacctgaaaaattcgcaccgcttgtcaaagagataaaaaagcatttgctcaagcggggcggctggagcataaaagatgtccttccgatccgaaaactccctaaaacaaccagcggaaaggttaaacgctacgaacttgccagacagtatgaggcagggaatttttcaacagagtctgccgccatcaatgaatatttggagagcagcccggaaacgtccggacagactcccattcatgaaattgaaacggaattactgtctatcttttccgatgtgctcaatgggaaaaaggttcacctcgctgacagttattttgatatgggagcaaattcattacagttatcgcagattgccgagcgcatagaacagaaattcggacgcgagcttgccgtttcagatctctttacgtatccttctatcactgatttagcggcgtatctgtctgaaagccgggctgaaatcaagcaggacgtggcagctaaaccaagccatgtgacaccgaaagatatcgccattatcgggatgtcgctcaatgtccctggagcatcaactaaaaatgatttttggaatctgcttgaaaaaggtgagcacagcattcgagaataccctgcatcccggctgaaagatgcggcggattatttaaagtccatccaaagcgaaatcaatgagaatcagtttgtgaagggcggctatttagatgaaatcgaccgctttgatttctcgttcttcggtttagctcctaaaacggctcagtttatggaccctaaccaaagactgtttttgcagtctgcatggcatgcgattgaagatgcgggctatgccggcggcagcatgaacgggagccgtgtcggggtatatgcagggtactcgaaggtgggctacgattatgaacgtctcctttctgcgaattatccggaggagcttcatcaatatatcgtgggcaatctcccttccgtgttagccagccgaatcgcttatttcttaaatttaaaagggccggcggtcacagtcgatacggcgtgctcctcatcgcttgccgccgttcatatggcatgtaaatctttaatatccggcgattgtgaaatggctcttgccggcggtatccggacatcgctcttgccgatctgtatcggacttgatatggaatcttcggacgggtacacgaaaacgttcagcaaagattcagacggtactggcacaggtgaaggcgcggccgcagtcctgctgaaacctctgcaggatgctgttcgcgacggagaccatatttacggcgtaatcaagggaagcgcgttgaatcaagacggaacaaccgccgggattacagcaccgaatccggcagctcagactgaggtcattgagacggcctggaaagacgcgggcattgcccctgaaacactgtctttcatcgaagcgcatggcaccggaacgaagctcggcgatccggttgaatttaacgggctttgtaaagcgtttgaaaagtatacggcaaaaaaacaattttgtgcgattggttctgttaaatcgaacatcggtcatttgtttgaagcggcaggcatcgtcgggctgatcaaatctgtcctcatgctgaatcacaagaaaaatccgccgttagtgcactttaatgaacctaatccgctcattcattttcactcttcaccattttacgtaaaccaggaagctgcagcgttcccatccggtgatgagccgctgcgaggcggagtcagctcatttggctttagcggaacgaacgctcatgtggtattggaagaatatatttctcaaagtgagtatgcgcccgaggatgaacatgggccgcacctatttgttttatccgctcatactgaaaaatcactctatgaactcgcacagcagtaccggcaatatgtatcggatgacagccaagcttcattaaagtccatttgctatacagccagtacgggcagggctcatttggatcatggcattgccatgattgtatccggtaaacaagaactatcggataagctgacccgcctgattcagggagacagaaaccttcccggtgtatacatcggctacaagaatatgaaggaaatgctgcccgctcataaagaagagctgaataaacaagcagccgcactgattaagcagcgtttacgtacgcaagatgaacggatcacatggctgcatcgcgccgccgaattatttgtgcaaggagccgttatcgattggcgcgcgctttattcaggtgaaactgtacaaaagacgccattgcccttgtatccgtttgaacggagccgatgctgggctgaagctgaccaattgcgcttaaacgaggacgaaaagagaggagaagcggcattgaatatcaatcaatcgaagtcgcatattgaatccttcctgaaaactgtaatcagcaatacttcggggatcagagcggaggaactcgatctgaatgctcattttatcggactcggaatggattctatcatgctgtcacaggtcaaaaaagccatcgcggacgaatttggggcagacatcccgatggatcgtttttttgatacgatgaacaaccttcaaagtgtcatagattacttggctgagaccgttccaacgtcctttgcatccgctccgcctcaagagaatgttccggcgcaggaaatgcaggtcatttcagaagcacagtctgaatcggatcgcagagaaggtcatcaagagcatatgctcgaaaaaataatcgcttctcagaatcaattaattcaggataccttgcaagctcaattaaatagctttaatttgttgagaaacagcggacatcattccgatgagaaagaatacgctaaagcgcaagagagatcaattccttctgtccagcaggggcctccggccgtcactgcagaaaagaaagcggctcaagaagcgaaaccctatgttcctttccagcctcagaacctgcatgaacagggacactataccgcacggcaaaaacaatacttagaagatttcatcaagaaatacgcagataaaacgaaaggttccaaacaatatacggacaacacccgatttgctcatgcaaacaaccgcaacttgtccagcttccgttcatattggaaggaaatcgtatacccgattatcgccgaacgttctgacggttctaaaatgtgggatattgacggaaatgaatatatcgatgtcaccatgggattcggggttaaccttttcgggcatcatccttcctttattacacaggttatcgatgattcagcccgttcttcattgcctccgctcggaccgatgtcagatgtcgccggtgaagttgccgaccggatccgcacatgtaccggggtagaaagggtcgctttctataattccggaacagaggccgtcatggttgccctgcgtttggcgcgggcggcaacaggaagaaagaaagtggtggcgttctcgggctcttatcacggcacgtttgacggcgtattaggggttgccggcacaaaaggcggagctgcgtctgcgaatccgctggctcctggtatactgcagagctttatggatgatttgattattttacattacaacaatcccgattctctggacgtgatccgcagtcttggtgatgaattggcagccgtactggtggaaccggtacaaagccgcagaccggatttgcagccgcgggcatttttgaaagaattgcgggcgatcacgcagcaatccggaacagctctgattatggatgaaattattaccggatttcggatcggtctcggcggcgcacaggaatggttcggcattcaggctgatttagtgacctacggaaaaatcatcggcggcggacagccgttaggggtagttgccggaaaagctgagttcatgaatgcgatcgacgggggtacctggcagtatggggacgattcctacccgcaagacgaggcgaaacgcacgtttgtggccggaaccttcaatactcatccgcttaccatgagaatgtcattagccgtgcttcgtcatttacaaaccgagggagaacatctgtatgagcagcttaatcaaaaaacagcctacttggtggatgagctgaatcgctgcttcgaacaagcgcaagtgcctatccgcatggttcgattcggttctttattccggtttgtctcatcgcttgataatgacttgttcttttaccatctcaactataaaggtgtctatgtgtgggaaggacgcaactgcttcttgtctgcggcgcataccgctgatgatatcgaaaagattattcaagcggtgaaagacacggtggaggatcttcgccgaggcggatttattccggaaggcccggactcccctgatggcggaggccgtaaaaagtccgggacgcgcgagctttcacctgaacaaaagcagttggttatggcatcccattacgggaatgaagcgtccgccgctttaaaccagtccattatgctgaaagtggagggcgaactgcagcatacaccattaaaacaagccgtccggcatatcgttggccgtcatgaagctttacgtacggtgattcatcccgatgacgaggtacagcaagtgcaggaacggatgaatatagaaataccagtcattgattttaccgttcacccgcatgaacatcgggagtcggaaattcaaaaatggctgacagaagatgccaagcggccgttccatttccatgaacaaaagcctttgtttagaatccatgtgcttacatcggctcacaatgaacatctgattgtgctcacgttccatcatatcattgccgatggatggtcaatcgccgtatttgttcaagaactggagagcaactacgcggcaatcgtacaaggaaaaccgatttcaccgaaagaggcagatgtttcgtttcgccaatacttagactggcagcaggcacagattgacagcggccattatgaagaaggggtccgttattggcggcgtcatttctctgaaccgattcagcagccaattctgccgagcacaggttctgtccgttatccgaacgggtatgagggagaccggtgtaccgtcaggcttggacggccattgagcgaggctttaaggtcattaagcattcagatgaaaaatagcgtatttgtgacaatgctgggtgcatttcatctttttctgcaccggcttaccaaacagtcaggccttgtgatcgggatccccgcagcaggtcaatcgcatataaaacagcatgatctgattggaaattgcgtcaatatgattccggtgaagaacacgtctacttcagaaagcactttaaccggttatcttggcagtatgaaagaaagcgtgaatcttgcaatgcggcaccaagccgtcccgatgacactggtggccagagagcttccgcacgatcaagtgccggatatgcgtattatctttaatttagacaggccttttcgaaagctgcatttcggaaaggcggaagcggagcccgttgcatacccggtaaaatgcaccctgtacgatttatttcttaacataacagacgcgcatcaagaatatgttcttgatttcgactttaatacgaacgtcatcagtccggaaatcatgaaaaagtggggagcgggttttacaaatttgctgcaaaaaatggttgagggggattcaatccctcttgacgccatgatgatgttttccgatgaagaacagcatgatttacaaaaactgtatgccgaacaccagaagcgggtctcttcaataggaagcaatacagcaaatttcactgaagcctacgaggcgccgataaatgaaacggaacggcagctggcgcggatttgggaggaacttttcggccttgaacgggtcggcagatcagatcgctttctggctctgggaggaaactcgctccaggcgacgcttatgctttccaaaattcagaagacatttcatcaaaaggtttccatcggacaatttttcaatcaccagactgttaaggaattagcacatttcattcagaacgaaacaaaagtcgtgcacctcccgatgaaagctgccgagaaaaaagcgtattacccgacatcgccggcgcagcaaagagtatatttcctgcaccaactggaaccggatcagctggcgcaaaatatgttcggccaaatatcaataacagggaagtacgatgagcaagccctgatctcatctcttcaacaagtgatgcagcggcacgaagcgtttcgcacgtatttcgacattatagatggcgatatcgttcagaaacttgaaaacgaagttgattttaacgttcatgtccggacaatgagccgggacgaatttgatgcctattcagaccggtttgtaaaaccgttccgcctggaccaagctccgttagttcgtgcggagctgatcaagattgaaaacgagcaggccgaactgctcatcgatatgcatcatatcatttcggatggttattccgtcaacatccttacaaatgaattgctggctttatatcatcagaaaccattaccggacattgaatttgaatataaagatttcgcagaatggcaaaaccaacggctgaatgaggatgccatgaagcggcaggagacatattggctggaacaatttcaagacgaaattcccatccttgacctgccgacagacggttcaaaagcggcagaacggtcttctgagggacagcgtgtgacatgctccttacagccggatgtcatccgttcgcttcaagatttggcgcaaaaggcggaaaccactctctatacggtgcttctggccgcctataatgtgctgcttcataaatataccggacaagaagacattgtcgtaggcacgcctgcttcaggaagaaatcatccggatatcgaaaaaatcatcggtattttcatacaaaccatcggaatccggacgaagccgcacgccaatagaacgtttacggattatctggaagaagtaaagcggcagacgcttgacgctttcgaaaaccaagactatccattcgaccggcttgtggagaaattaaatgtgcagcgggaaacaaccggaaagtctctgtttaacacgatgtttgtgtttcaaaacattgaatttcatgaaatccggcacaatgaatgtacatttaaagtgaaagaacgaaatccaggggtctctttgtatgatttgatgctcacgatcgaagatgccggtcaacagatagagatgcattttgattataaaccgggacgattcacaaaagacaccattgaacagatcaccagacactataccggcattttaaacagtcttgttgagcagccggagatgacattgtcttccgttcctatgctgtctgaaaccgaacggcatcaactgctgacggagtgtaacggcacaaagacgccgtatccgcataacgaaacagtaacccgatggtttgaaatgcaggcggaacagagtcccgatcatgcagccgttatttttggcaatgagcggtatacgtacagacagctcaatgaacgggcgaaccgattggcgcggacgttacggacaaaaggcgtacaagcggatcaattcgttgccatcatctctccgcatcgcatcgagttgattgttggtattttggctgttctgaaatcaggcggcgcatacgtgcctattgatcctgaatatccggaagatcggatccaatatatgctgagagattcaagggcggaggttgtgttgacacagcgcagcctgctggatcaattaccgtatgatggtgacgttgtgcttttggatgaggaaaactcataccatgaggatcactcgaatcttgaatcggacagcgatgcgcatgatttggcctacatgatctatacgtcaggttccacgggaaatccaaaaggtgtcctcattgagcatcagggactggctgattatatttggtgggcgaaagaggtttatgtaagaggtgagaaaaccaacttcccattatactcttccatctctttcgatctgactgtgacctcgatatttacaccgctggttacgggaaataccatcattgtctttgacggcgaagacaaaagcgctgtgctttctgagattatgcgggactcaagaatagacatgatcaaattgaccccggcacatctgcacgtcatcaaggagatgaatatcggtggcggcaccgcaatacggaaaatgattgtcggcggagaaaatttaagcacccgtctggccaaaagtgtcagcgagcagtttaaaggccggctggacattttcaatgaatacggaccgacggaagctgtcgtcggatgtatgatttatcacttcgacgcagaacgggacaagcgggaatttgtaccgatcggcactccggctgccaacacggatatttatgtggccgatgcaagcagaaatctggttccgatcggggtaatcggcgaaatatatatcagcggaccgggtgttgccagagggtattggaaccgtccggatttaacggcagagaaatttgttgaaaacccgtatgtcccgggagcgaagatgtacaaatcaggggatttggctaagcggttgaaggacggaaaccttgtatatatcgggcgcgttgatgaacaagtcaaaatcaggggataccgaatcgagcttggtgaaattgaagcagcaatgcataacgcggaagcggtgcaaaaagccgcggttacagtgaaagaagaagaagacggcttaaaacaattatgcgcgtattacgtaagcgacaagcctatagcggctgcgcagcttagggaacaattgtcatcggagcttccggactacatggttccgtcctattttgtccaactggagcatatgccgttaacgtccaacgggaaaataaaccgtaaggcactgccagcaccagaagcgagtctgcagcagacagctgaatatgttccgccgggtaatgagacggagtccaaactgacagatttatggaaggaagtgctcggaataagccatgcggggatcaaacataatttctttgatctcggaggcaactccatccgagcggctgccttggccgccagaattcacaaagagctggatgtgaatctgtctctcaaagacatattcaagtttcctaccattgaacaattggctgacaaggcgttacacatggacaaaaaccgatatgtaccgattccggctgcaaaggaaatgccatattatccggtttcttcagctcagaggcgtatgtatttgttaagtcacacagaaggcggcgagctgacttacaatatgacgggtgccatgaatgtggaagggacgatcgatcccgaacggttaaacgccgctttccgaaaattaatcgcgcgtcatgaagcgttgcggaccagctttgatttatatgaaggcgagccggcacagcgtattcatcagaacgtcgactttacgatagaacggattcaagcaagcgaagaagaagcggaagaccgtgtgcttgatttcatcaaagcgtttgacttagccaaaccgccgctgatgcgggccggactgattgaaattgaacctgcgcggcacgtgcttgtggttgatatgcatcatatcatttctgacggcgtgtccgtcaatattctgatgaaagatttaagccgaatctacgaggggaacgaaccggacccgctctctattcaatataaagactttgcagtttggcagcaatcggacattcagaaacggaacatcaagagccaggaagcgtattggctggatcagtttcacagtgatattcctgtactggatatgcctgcggattatgagagacctgccatacgcgattacgaaggcgaatcatttgaatttcttatacccgaacacttgaaacagcgtttaagccaaatggaagaagacacaggagcaacactgtatatgattttattggcggcctatacgattcttttatccaggtacagcggacaagaagatattatcgtaggaacgccatctgccgggcggactcatttggatgtagagccggtcgtgggaatgttcgtcaatacgttagtcattcgcaatcacccggcgggccgtaaaacatttgatgcctacttaaacgaagtaaaagaaaacatgctgaacgcctataaaaatcaagactatccattggaagaattaattcagcatctgcatcttccaaaagattcaagccgcaatcctttattcgatacgatgtttgtgctgcaaaatctcgatcatgctgaattgactttcgattctcttcaactcaagccgtattcatttcatcatccggttgccaaattcgatttgaccttgtcgattcaggcggaccaagacaactatcacggactgtttgaatattcgaaaaaactgtttaagaaaagcagaatcgaggttttatcaaacgactacttacacattctatcggcgattttggaacaaccaagcattctaattgaacatatcggattgagcggcagcaatgaggaagaagagaacgcgcttgattctattcaattgaacttttag

SEQ ID NO: 9 - protein, 400 aa, Bacillus amyloliquefaciens
MNNLAFLFPGQGSQFVGMGKQFWNDFVLAKRLFEEASDAISLDVKKLCFNGDMNELTKTMNAQPAILTVSVIAFQVYMQEIGVKPRFLAGHSLGEYSALVCAGALSFQDAVTLVRQRGILMQNADPQQQGTMAAVTHLSLQTLQEICSKVSTEDFPAGVACMNSEQQHVISGHRQAVERVIKMAEEKGAAYTYLNVSAPFHSSLIRSASEQFQTVLHRYSFRDAAWPIISNVTARPYSSGNSISEHLEQHMTMPVRWTESMHYLLLHGVTEVIEMGPNNVLAGLLRKTTNHIVPYPLGQTSDVHLLSNSAERKKHIVRLRKKQLNKLMIQSVIARNYNKDSAAYSNMTTALFTQIQELKERMERHENELSEQELEHSIHLCKLICEAKQLPDWEELRILK

SEQ ID NO: 10 - protein, 3982 aa, Bacillus amyloliquefaciens
MYTSQFQTLVDVIRERSNISDRGIRFIESDKNETVVSYRQLFEEAQGYLGYLQHIGIQPKQEIVFQIQENKSFVVAFWACILGGMIPVPVSIGEDDDHKLKVWRIWNILNHPFLIASEKVLDKIKKYAAEHDLQDFHHQLNEKSDIIQDQTYDYPASFYEPDADELAFIQFSSGSTGDPKGVMLTHHNLIHNTCAIGNALAVHSRDSFLSWMPLTHDMGLIACHLVPFITGINQNLMPTELFIRRPILWMKKAHEHKASILSSPNFGYNYFLKFLKNEPDWDLSHIKVIANGAEPILPELCDEFLKRCAAFNLKRSAILNVYGLAEASVGAAFSKLGKEFVPVYLHRDHLNLGERAVNVSKEDQNCASFVEVGRPIDYCQLRISDEANERVEDGIIGHIQIKGDNVTQGYYNNPESTEKALTSDGWVKTGDLGFISESGNLVVTGREKDIIFVNGKNIYPHDIERVAIEMEEVDLGRVAACGVYDQKTQSGEIVLFVVYKKSPEKFAPLVKEIKKHLLKRGGWSIKDVLPIRKLPKTTSGKVKRYELARQYEAGNFSTESAAINEYLESSPETSGQTPIHEIETELLSIFSDVLNGKKVHLADSYFDMGANSLQLSQIAERIEQKFGRELAVSDLFTYPSITDLAAYLSESRAEIKQDVAAKPSHVTPKDIAIIGMSLNVPGASTKNDFWNLLEKGEHSIREYPASRLKDAADYLKSIQSEINENQFVKGGYLDEIDRFDFSFFGLAPKTAQFMDPNQRLFLQSAWHAIEDAGYAGGSMNGSRVGVYAGYSKVGYDYERLLSANYPEELHQYIVGNLPSVLASRIAYFLNLKGPAVTVDTACSSSLAAVHMACKSLISGDCEMALAGGIRTSLLPICIGLDMESSDGYTKTFSKDSDGTGTGEGAAAVLLKPLQDAVRDGDHIYGVIKGSALNQDGTTAGITAPNPAAQTEVIETAWKDAGIAPETLSFIEAHGTGTKLGDPVEFNGLCKAFEKYTAKKQFCAIGSVKSNIGHLFEAAGIVGLIKSVLMLNHKKNPPLVHFNEPNPLIHFHSSPFYVNQEAAAFPSGDEPLRGGVSSFGFSGTNAHVVLEEYISQSEYAPEDEHGPHLFVLSAHTEKSLYELAQQYRQYVSDDSQASLKSICYTASTGRAHLDHGIAMIVSGKQELSDKLTRLIQGDRNLPGVYIGYKNMKEMLPAHKEELNKQAAALIKQRLRTQDERITWLHRAAELFVQGAVIDWRALYSGETVQKTPLPLYPFERSRCWAEADQLRLNEDEKRGEAALNINQSKSHIESFLKTVISNTSGIRAEELDLNAHFIGLGMDSIMLSQVKKAIADEFGADIPMDRFFDTMNNLQSVIDYLAETVPTSFASAPPQENVPAQEMQVISEAQSESDRREGHQEHMLEKIIASQNQLIQDTLQAQLNSFNLLRNSGHHSDEKEYAKAQERSIPSVQQGPPAVTAEKKAAQEAKPYVPFQPQNLHEQGHYTARQKQYLEDFIKKYADKTKGSKQYTDNTRFAHANNRNLSSFRSYWKEIVYPIIAERSDGSKMWDIDGNEYIDVTMGFGVNLFGHHPSFITQVIDDSARSSLPPLGPMSDVAGEVADRIRTCTGVERVAFYNSGTEAVMVALRLARAATGRKKVVAFSGSYHGTFDGVLGVAGTKGGAASANPLAPGILQSFMDDLIILHYNNPDSLDVIRSLGDELAAVLVEPVQSRRPDLQPRAFLKELRAITQQSGTALIMDEIITGFRIGLGGAQEWFGIQADLVTYGKIIGGGQPLGVVAGKAEFMNAIDGGTWQYGDDSYPQDEAKRTFVAGTFNTHPLTMRMSLAVLRHLQTEGEHLYEQLNQKTAYLVDELNRCFEQAQVPIRMVRFGSLFRFVSSLDNDLFFYHLNYKGVYVWEGRNCFLSAAHTADDIEKIIQAVKDTVEDLRRGGFIPEGPDSPDGGGRKKSGTRELSPEQKQLVMASHYGNEASAALNQSIMLKVEGELQHTPLKQAVRHIVGRHEALRTVIHPDDEVQQVQERMNIEIPVIDFTVHPHEHRESEIQKWLTEDAKRPFHFHEQKPLFRIHVLTSAHNEHLIVLTFHHIIADGWSIAVFVQELESNYAAIVQGKPISPKEADVSFRQYLDWQQAQIDSGHYEEGVRYWRRHFSEPIQQPILPSTGSVRYPNGYEGDRCTVRLGRPLSEALRSLSIQMKNSVFVTMLGAFHLFLHRLTKQSGLVIGIPAAGQSHIKQHDLIGNCVNMIPVKNTSTSESTLTGYLGSMKESVNLAMRHQAVPMTLVARELPHDQVPDMRIIFNLDRPFRKLHFGKAEAEPVAYPVKCTLYDLFLNITDAHQEYVLDFDFNTNVISPEIMKKWGAGFTNLLQKMVEGDSIPLDAMMMFSDEEQHDLQKLYAEHQKRVSSIGSNTANFTEAYEAPINETERQLARIWEELFGLERVGRSDRFLALGGNSLQATLMLSKIQKTFHQKVSIGQFFNHQTVKELAHFIQNETKVVHLPMKAAEKKAYYPTSPAQQRVYFLHQLEPDQLAQNMFGQISITGKYDEQALISSLQQVMQRHEAFRTYFDIIDGDIVQKLENEVDFNVHVRTMSRDEFDAYSDRFVKPFRLDQAPLVRAELIKIENEQAELLIDMHHIISDGYSVNILTNELLALYHQKPLPDIEFEYKDFAEWQNQRLNEDAMKRQETYWLEQFQDEIPILDLPTDGSKAAERSSEGQRVTCSLQPDVIRSLQDLAQKAETTLYTVLLAAYNVLLHKYTGQEDIVVGTPASGRNHPDIEKIIGIFIQTIGIRTKPHANRTFTDYLEEVKRQTLDAFENQDYPFDRLVEKLNVQRETTGKSLFNTMFVFQNIEFHEIRHNECTFKVKERNPGVSLYDLMLTIEDAGQQIEMHFDYKPGRFTKDTIEQITRHYTGILNSLVEQPEMTLSSVPMLSETERHQLLTECNGTKTPYPHNETVTRWFEMQAEQSPDHAAVIFGNERYTYRQLNERANRLARTLRTKGVQADQFVAIISPHRIELIVGILAVLKSGGAYVPIDPEYPEDRIQYMLRDSRAEVVLTQRSLLDQLPYDGDVVLLDEENSYHEDHSNLESDSDAHDLAYMIYTSGSTGNPKGVLIEHQGLADYIWWAKEVYVRGEKTNFPLYSSISFDLTVTSIFTPLVTGNTIIVFDGEDKSAVLSEIMRDSRIDMIKLTPAHLHVIKEMNIGGGTAIRKMIVGGENLSTRLAKSVSEQFKGRLDIFNEYGPTEAVVGCMIYHFDAERDKREFVPIGTPAANTDIYVADASRNLVPIGVIGEIYISGPGVARGYWNRPDLTAEKFVENPYVPGAKMYKSGDLAKRLKDGNLVYIGRVDEQVKIRGYRIELGEIEAAMHNAEAVQKAAVTVKEEEDGLKQLCAYYVSDKPIAAAQLREQLSSELPDYMVPSYFVQLEHMPLTSNGKINRKALPAPEASLQQTAEYVPPGNETESKLTDLWKEVLGISHAGIKHNFFDLGGNSIRAAALAARIHKELDVNLSLKDIFKFPTIEQLADKALHMDKNRYVPIPAAKEMPYYPVSSAQRRMYLLSHTEGGELTYNMTGAMNVEGTIDPERLNAAFRKLIARHEALRTSFDLYEGEPAQRIHQNVDFTIERIQASEEEAEDRVLDFIKAFDLAKPPLMRAGLIEIEPARHVLVVDMHHIISDGVSVNILMKDLSRIYEGNEPDPLSIQYKDFAVWQQSDIQKRNIKSQEAYWLDQFHSDIPVLDMPADYERPAIRDYEGESFEFLIPEHLKQRLSQMEEDTGATLYMILLAAYTILLSRYSGQEDIIVGTPSAGRTHLDVEPVVGMFVNTLVIRNHPAGRKTFDAYLNEVKENMLNAYKNQDYPLEELIQHLHLPKDSSRNPLFDTMFVLQNLDHAELTFDSLQLKPYSFHHPVAKFDLTLSIQADQDNYHGLFEYSKKLFKKSRIEVLSNDYLHILSAILEQPSILIEHIGLSGSNEEEENALDSIQLNF

SEQ ID NO: 11 - protein, 10 aa, Bacillus subtilis
  misc_feature (2)..(2): Xaa is Orn
EXYTEAPQTI

SEQ ID NO: 12 - protein, 10 aa, Bacillus subtilis
  misc_feature (2)..(2): Xaa is Orn
EXYTEVPQTI

SEQ ID NO: 13 - protein, 10 aa, Bacillus subtilis
  misc_feature (2)..(2): Xaa is Orn
EXYTEAPQYI

SEQ ID NO: 14 - protein, 10 aa, Bacillus subtilis
  misc_feature (2)..(2): Xaa is Orn
EXYTEVPQYI

SEQ ID NO: 15 - protein, 7 aa, Bacillus subtilis
ELLVDLL

SEQ ID NO: 16 - protein, 7 aa, Bacillus subtilis
  misc_feature (1)..(1): Xaa is Gln or Glu
  misc_feature (2)..(2): Xaa is Leu or ILe
  misc_feature (4)..(4): Xaa is Val or ILE
  misc_feature (7)..(7): Xaa is Val or ILE
XXLXDLX

SEQ ID NO: 17 - protein, 7 aa, Bacillus subtilis
  misc_feature (7)..(7): Xaa is VAL or ILE
ELLLDLX

SEQ ID NO: 18 - protein, 7 aa, Bacillus subtilis
  misc_feature (2)..(2): Xaa is Val, Leu or ILe
  misc_feature (4)..(4): Xaa is Ala, Val, Leu or ILe
  misc_feature (7)..(7): Xaa is Val, Leu or ILe
EXLXDLX

SEQ ID NO: 19 - protein, 7 aa, Bacillus subtilis
NYNQPNS

SEQ ID NO: 20 - protein, 7 aa, Bacillus subtilis
NYNQPNS

SEQ ID NO: 21 - protein, 7 aa, Bacillus subtilis
NYNQPNS

SEQ ID NO: 22 - protein, 7 aa, Bacillus subtilis
NYNQPSN

SEQ ID NO: 23 - protein, 7 aa, Bacillus subtilis
NYNPEST

SEQ ID NO: 24 - protein, 7 aa, Bacillus subtilis
NYNQPNT

SEQ ID NO: 25 - protein, 7 aa, Bacillus subtilis
NYNSQST

SEQ ID NO: 26 - protein, 7 aa, Bacillus subtilis
NYNSEST

SEQ ID NO: 27 - protein, 8 aa, Bacillus subtilis
  misc_feature (6)..(6): xaa is betaAA
NSESTXNY